(19) United States
(12) Patent Application Publication
(10) Pub. No.: US 2018/0300506 A1
(43) Pub. Date: Oct. 18, 2018

(54) INFORMATION PROCESSING APPARATUS AND INFORMATION PROCESSING SYSTEM

(71) Applicant: FUJITSU LIMITED, Kawasaki-shi (JP)

(72) Inventors: Toru KAWAKAMI, Yokohama (JP); Akito YAMAZAKI, Kawasaki (JP); Kazunori Kobashi, Yamato (JP)

(73) Assignee: FUJITSU LIMITED, Kawasaki-shi (JP)

(21) Appl. No.: 15/925,853

(22) Filed: Mar. 20, 2018

(30) Foreign Application Priority Data
Apr. 17, 2017 (JP) 2017-081245

(51) Int. Cl. G06F 21/62; H04L 9/06

(52) U.S. Cl. CPC G06F 21/6254; G16H 10/60; H04L 9/0643

(57) ABSTRACT

An information processing apparatus includes a memory and a processor coupled to the memory and configured to receive a first request provided from a first device that requests personal information associated with a first identifier, the first request including a second identifier obtained by converting the first identifier, and a second request which includes an extraction condition and is provided from a second device that requests concealed personal information corresponding to the extraction condition, extract personal information associated with the second identifier from a storage device, when the first request is received, and extract concealed personal information corresponding to the extraction condition from the storage device, when the second request is received; and transmit the extracted personal information to the first device, when the first request is received, and transmit the extracted concealed personal information to the second device, when the second request is received.

### CROSS-REFERENCE TO RELATED APPLICATION

This application is based upon and claims the benefit of priority of the prior Japanese Patent Application No. 2017-081245, filed on Apr. 17, 2017, the entire contents of which are incorporated herein by reference.

### FIELD

The embodiments discussed herein are related to an information processing apparatus, an information processing system, and a non-transitory computer-readable recording medium having stored therein a program.

### BACKGROUND

In recent years, the need for big data analysis is increasing more and more. For the big data analysis, it is desirable to collect data samples as many as possible in order to obtain more accurate and useful analysis results. The importance of data is increasing more and more with the development of Artificial Intelligence (AI) and Internet of Things (IoT). Therefore, it is believed that countries and companies with more data will become dominant in the future competitive society, and the government is also working on promoting the utilization of medical data.

There may be a plan on a government level to enforce a future policy to promote big data analysis in the domestic medical field. According to this policy, data on hospital electronic clinical charts (electronic medical records) are collected and processed into anonymous data which are to be provided to organizations that wish to utilize the anonymous data as data available for the big data analysis. In addition, for personalized services such as healthcare services, in order to refer to, for example, the service user's vital data or hospital history, the user's data that are not concealed (non-concealed data) are scheduled to be provided under the consent of the service user.

The electronic clinical charts are data containing a lot of personal information related to patient's privacy. For this reason, when collecting such data in large quantities, it is desirable to take measures to prevent the leakage of personal information.

Various techniques for collecting and utilizing medical data such as electronic clinical charts are also known.

Related techniques are disclosed in, for example, Japanese Laid-Open Patent Publication Nos. 2013-250754 and 2008-108021, and International Publication Pamphlet No. WO2008/069011.

### SUMMARY

According to an aspect of the embodiments, provided is an information processing apparatus including a memory and a processor coupled to the memory and configured to receive, from a management device, a first request provided from a first device that requests personal information associated with a first identifier, the first request including a second identifier obtained by converting the first identifier, and a second request which includes an extraction condition and is provided from a second device that requests concealed personal information corresponding to the extraction condition, extract personal information associated with the second identifier from a storage device that stores a plurality of personal information associated respectively with a plurality of second identifiers, when the first request is received, and extract concealed personal information corresponding to the extraction condition from the storage device that stores a plurality of concealed personal information, when the second request is received, and transmit the extracted personal information associated with the second identifier to the first device, when the first request is received, and transmit the extracted concealed personal information corresponding to the extraction condition to the second device, when the second request is received.

The object and advantages of the invention will be realized and attained by means of the elements and combinations particularly pointed out in the claims. It is to be understood that both the foregoing general description and the following detailed description are exemplary and explanatory and are not restrictive of the invention, as claimed.

### BRIEF DESCRIPTION OF DRAWINGS

FIG. 1 is a view illustrating the configuration of an information processing system according to an embodiment;

FIG. 2 is an example of a clinical chart information DB;

FIG. 3 is an example of a personal management DB;

FIG. 4 is an example of a patent management DB;

FIG. 5 is an example of a hospital management DB;

FIG. 6 is an example of a collection system management DB;

FIG. 7 is an example of an anonymous ID clinical chart information DB;

FIG. 8 is an example of a temporary DB generated from the anonymous ID clinical chart information DB;

FIG. 9 is an example of a concealment clinical chart information DB;

FIG. 10 is an example of a temporary DB generated from the concealment clinical chart information DB;

FIG. 11 is a view illustrating a data collection process according to an embodiment;

FIG. 12A is a sequence diagram of a data collection process according to an embodiment;

FIG. 12B is a sequence diagram of a data collection process according to an embodiment;

FIG. 12C is a sequence diagram of a data collection process according to an embodiment;

FIG. 13 is a view illustrating a concealment process according to an embodiment;

FIG. 14 is a sequence diagram of a concealment process according to an embodiment;

FIG. 15 is a view illustrating a process of transmitting non-concealed data to a service utilization system according to an embodiment;

FIG. 16A is a sequence diagram of a process of transmitting non-concealed data to a service utilization system according to an embodiment;

FIG. 16B is a sequence diagram of a process of transmitting non-concealed data to a service utilization system according to an embodiment;

FIG. 16C is a sequence diagram of a process of transmitting non-concealed data to a service utilization system according to an embodiment;

FIG. 16D is a sequence diagram of a process of transmitting non-concealed data to a service utilization system according to an embodiment;

FIG. 16E is a sequence diagram of a process of transmitting non-concealed data to a service utilization system according to an embodiment;

FIG. 16F is a sequence diagram of a process of transmitting non-concealed data to a service utilization system according to an embodiment;

FIG. 16G is a sequence diagram of a process of transmitting non-concealed data to a service utilization system according to an embodiment;

FIG. 16H is a sequence diagram of a process of transmitting non-concealed data to a service utilization system according to an embodiment;

FIG. 16I is a sequence diagram of a process of transmitting non-concealed data to a service utilization system according to an embodiment;

FIG. 16J is a sequence diagram of a process of transmitting non-concealed data to a service utilization system according to an embodiment;

FIG. 17 is a view illustrating a process of transmitting non-concealed data to a research institution system according to an embodiment;

FIG. 18A is a sequence diagram of a process of transmitting non-concealed data to a research institution system according to an embodiment;

FIG. 18B is a sequence diagram of a process of transmitting non-concealed data to a research institution system according to an embodiment;

FIG. 18C is a sequence diagram of a process of transmitting non-concealed data to a research institution system according to an embodiment;

FIG. 18D is a sequence diagram of a process of transmitting non-concealed data to a research institution system according to an embodiment;

FIG. 18E is a sequence diagram of a process of transmitting non-concealed data to a research institution system according to an embodiment;

FIG. 18F is a sequence diagram of a process of transmitting non-concealed data to a research institution system according to an embodiment;

FIG. 18G is a sequence diagram of a process of transmitting non-concealed data to a research institution system according to an embodiment;

FIG. 18H is a sequence diagram of a process of transmitting non-concealed data to a research institution system according to an embodiment; and

FIG. 19 is a view illustrating the configuration of an information processing apparatus (computer).

### DESCRIPTION OF EMBODIMENTS

As described above, for personalized services such as healthcare services, non-concealed data are scheduled to be provided under the consent of the service user.

However, there is a problem in applying a mechanism for providing anonymous data to research institutions such as universities based on the above-mentioned government policy to a usage form that provides non-concealed data to personalized services such as healthcare services.

In addition, managing data collected from each hospital based on patient common IDs in the one-to-one correspondence with information identifying individuals such as “MyNumber” involves risks.

This problem is not limited to the case where electronic clinical charts are used in hospitals, and may also occur in a case where other information is used in other information providing institutions.

Hereinafter, embodiments will be described with reference to the drawings. As the current situations of medical data utilization, although there are some hospitals and areas that introduce electronic clinical charts, the penetration rate is not so high. Furthermore, although electronic clinical charts are introduced, there is no mechanism that is enough to utilize these data.

So, the government considers the construction of “next generation medical ICT infrastructure” to be operated in the future. The “next generation medical IC infrastructure” is a mechanism in which a plurality of information collection agencies collect medical data from, for example, hospitals and provide the data to personalized services such as healthcare services and research institutions such as universities.

Personalized services such as healthcare services collect user personal data that are not concealed because they refer to, for example, user's vital data and hospital history under the consent of the service user. This is different from research institutions such as universities in terms of the purpose of using the data. A mechanism that provides concealed data to research institutions such as universities has a difficulty in coping with usage forms that provide data to personal services such as healthcare services.

In addition, when a process of concealing data collected from each hospital by a patient common ID linked one-to-one with information identifying a person such as “MyNumber,” managing the data with the same patient common ID all the time involves risks. For example, when there is a malicious person at a certain site of an information collection institution, since the patient common ID is associated one-to-one with the information identifying a person such as “MyNumber” and the information collection institution holds personal medical data, there is a problem that the malicious person may collect the specific personal information easily. In addition, since medical data of different hospitals are managed with the same patient common ID in order to identify the same person's data, there is a problem that information of specified persons between different hospitals may be merged from the patient common ID to obtain more detailed information. Furthermore, when there are malicious persons in a plurality of collection institutions, there is a problem that the malicious persons may obtain more detailed information by merging information of specific persons held by each other from the patient common ID.

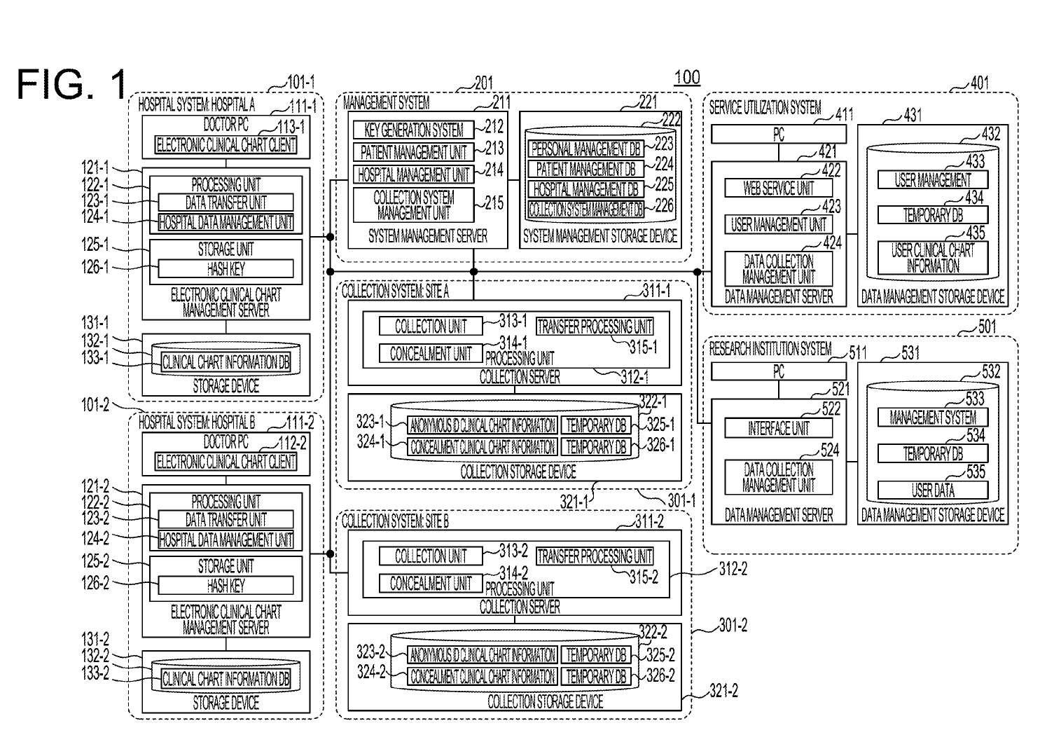

FIG. 1 is a view illustrating the configuration of an information processing system according to an embodiment. In the information processing system 100 of FIG. 1, an information providing institution is a hospital that provides data of electronic clinical charts (electronic medical records), an information management institution is an institution such as a government that manages user data. An information collection institution is an institution such as a government that collects data on electronic clinical charts. An information analysis institution is an institution such as a research institution, a pharmaceutical company or the like that analyzes and uses data of concealed electronic clinical charts. A service utilization institution is an institution that uses data of non-concealed electronic clinical charts to provide healthcare services. The electronic clinical charts are an example of personal information.

The information processing system 100 includes a hospital system 101-1 of a hospital A, a hospital system 101-2 of a hospital B, a management system 201 of an information management institution, a collection system 301-1 of a site A of an information collection institution, a collection system 301-2 of a site B of the information collection institution, a service utilization system 401 of an information utilization institution, and a research institution system 501 of an information analysis institution.

The number of hospital systems is not limited to two but may be three or more when the number of hospitals is three or more. For example, a plurality of hospitals existing nationwide may be information providing institutions. The number of collection systems is not limited to two but may be three or more when the number of sites is three or more. The number of collection systems may be one.

A hospital system 101-i (i=1, 2) includes a doctor personal computer (PC) 111-I, an electronic clinical chart management server 121-i, and a storage device 131-i. The hospital system 101-1 stores and manages patient electronic clinical charts of the hospital A, and the hospital system 101-2 stores and manages patient electronic clinical charts of the hospital B. The hospital system 101-i is an example of a personal information management apparatus.

An electronic clinical chart client 113-i as an application is installed in the doctor PC 111-i. The doctor PC 111-i is communicably connected to the electronic clinical chart management server 121-i via a network. Based on the operation by a doctor, the electronic clinical chart client 113-i inputs the patient data to an electronic clinical chart and transmits the data to the electronic clinical chart management server 121-i.

The electronic clinical chart management server 121-i is communicably connected to the doctor PC 111-i and the storage device 131-i via a network. The electronic clinical chart management server 121-i, a system management server 211, collection servers 312-1 and 312-2, and data management servers 421 and 521 are communicably interconnected via a network. The electronic clinical chart management server 121-i includes a processing unit 122-i and a storage unit 125-i. The electronic clinical chart management server 121-i is an example of an information processing apparatus.

The processing unit 122-i includes a data transfer unit 123-i and a hospital data management unit 124-i.

The data transfer unit 123-i uses a hash key 126-i to hash a personal ID to generate an anonymous personal ID. The data transfer unit 123-i transmits the anonymous personal ID and data of a predetermined item of a clinical chart information DB 133-i to one of the collection servers 312-1 and 312-2.

The hospital data management unit 124-i determines whether or not hospital information is registered. The storage unit 125-i stores the hash key 126-i which is a secret key used to calculate a hash value. The hash key 126-i has different values for different hospital systems 101-i. Therefore, different anonymous personal IDs are generated for different hospital systems 101-i for the same personal ID.

The storage device 131-i includes a storage unit 132-i. The storage unit 132-i stores the clinical chart information DB 133-i. The clinical chart information DB 133-i includes electronic clinical charts of a plurality of patients.

The management system 201 includes the system management server 211 and a system management storage device 221.

The system management server 211 includes a key generation unit 212, a patient management unit 213, a hospital management unit 214, and a collection system management unit 215. The system management server 211 is an example of an information processing apparatus.

The key generation unit 212 generates different hash keys 126-i for different hospital systems 101-i.

The patient management unit 213 manages information on users (or written as patients). The hospital management unit 214 manages information on hospitals.

The collection system management unit 215 manages information on the collection system 301-i. The system management storage device 221 includes a storage unit 222.

The storage unit 222 stores a personal management DB 223, a patient management DB 224, a hospital management DB 225, and a collection system management DB 226.

The personal management DB 223 indicates the correspondence relationship between a personal ID and a patient ID. The personal ID and the patient ID are unique identifiers (identification information) for identifying users, which are assigned for different users (patients). The personal ID is assigned to users by, for example, an institution such as a government. The patient ID is assigned to users by the system management server 211. One patient ID corresponds to one personal ID.

The patient management DB 224 indicates hospitals where patients are examined and diagnosed. That is, the patient management DB 224 indicates hospitals (the hospital systems 101-i) having electronic clinical charts of patients.

The hospital management DB 225 includes information (hospital information) on hospitals (the hospital systems 101-i).

The collection system management DB 226 includes information on the collection system 301-i. The personal management DB 223, the patient management DB 224, the hospital management DB 225, and the collection system management DB 226 will be described in more detail later.

The collection system 301-i includes a collection server 311-i and a collection storage device 321-i. The collection server 311-i is communicably connected to the collection storage device 321-i via a network.

The collection server 311-i includes a processing unit 312-i. The collection server 311-i is an example of an information processing apparatus.

The processing unit 312-i includes a collection unit 313-i, a concealment unit 314-i, and a transfer processing unit 315-i.

The collection unit 313-i receives electronic clinical charts from the hospital system 101-i. The concealment unit 314-i conceals data of a predetermined item of the electronic clinical charts collected by the collection unit 313-i.

The transfer processing unit 315-i transmits non-concealed data included in an anonymous ID clinical chart information DB 323-i to the data management server 421 of the service utilization system 401. The transfer processing unit 315-i transmits concealed data included in a concealment clinical chart information DB 324-i to the data management server 521 of the research institution system 501. The transfer processing unit 315-i is an example of a receiving unit, an extracting unit, and a transmitting unit.

The collection storage device 321-i includes a storage unit 322-i. The storage unit 322-i stores the anonymous ID clinical chart information DB 323-i, the concealment clinical chart information DB 324-i, and temporary DBs 325-i and 326-i.

The anonymous ID clinical chart information DB 323-i includes electronic clinical charts including hashed personal IDs (anonymous personal IDs). Data of predetermined items (date of birth, address, etc.) of the electronic clinical charts included in the anonymous ID clinical chart information DB 323-i are not concealed. The electronic clinical charts included in the anonymous ID clinical chart information DB 323-i are an example of non-concealed data.

The concealment clinical chart information DB 324-i includes concealed electronic clinical charts. Data of predetermined items (date of birth, address, etc.) of the electronic clinical charts included in the concealment clinical chart information DB 324-i are concealed. The electronic clinical charts included in the concealment clinical chart information DB 324-i are an example of concealed data.

The temporary DB 325-i is a temporarily generated database and includes data extracted from the anonymous ID clinical chart information DB 323-i.

The temporary DB 326-i is a temporarily generated database and includes data extracted from the concealment clinical chart information DB 324-i.

The service utilization system 401 includes a PC 411, a data management server 421, and a data management storage device 431.

The PC 411 uses a browser, a graphical user interface (GUI) or the like to request the data management server 421 to acquire the user's electronic clinical charts based on an operation by a user. In addition, the PC 411 may use a browser, a GUI or the like to request the data management server 421 to acquire the user's electronic clinical charts based on an operation by an operator under the consent of the user.

The data management server 421 requests the management system server 211 for the user's electronic clinical charts and stores the received electronic clinical charts in a user clinical chart information DB 435. The data management server 421 includes a web service unit 422, a user management unit 423, and a data collection management unit 424. The data management server 421 is an example of an information processing apparatus.

The web service unit 422 exchanges data with the PC 411. The user management unit 423 uses a user management DB 433 to perform management such as logging in the data management server 421.

The data collection management unit 424 receives data from the collection system 301-i and stores the data in a data management storage device 431.

The data management storage device 431 includes a storage unit 432. The storage unit 432 stores the user management DB 433, a temporary DB 434, and the user clinical chart information DB 435.

The user management DB 433 includes user information (account and password) of users who use the service utilization system 401 and personal IDs of the users.

The temporary DB 434 is a temporarily generated database and includes the user's electronic clinical charts received from the collection server 312-i. A plurality of temporary DBs 434 may be used.

The user clinical chart information DB 435 includes electronic clinical charts of users. The research institution system 501 includes a PC 511, a data management server 521, and a data management storage device 531.

Based on an operation of a researcher of the research institution system 501, the PC 511 inputs a condition and requests the data management server 421 to acquire a concealed electronic clinical chart matching the condition.

The data management server 521 includes an interface unit 522 and a data collection management unit 524. The data management server 521 is an example of an information processing apparatus.

The interface unit 522 communicates with the PC 511. The data collection management unit 524 requests the data management server 421 to provide a concealed electronic clinical chart matching an input filter condition.

The data management storage device 531 includes a storage unit 532. The storage unit 532 stores a management system DB 533, a temporary DB 534, and a user data DB 535.

The management system DB 533 contains information to be used for communication with the system management server 211. The management system DB 533 includes, for example, the domain name and the IP address of the system management server 211.

The temporary DB 534 is a temporarily generated database and includes a concealed electronic clinical chart matching to the input condition received from the collection server 312-i.

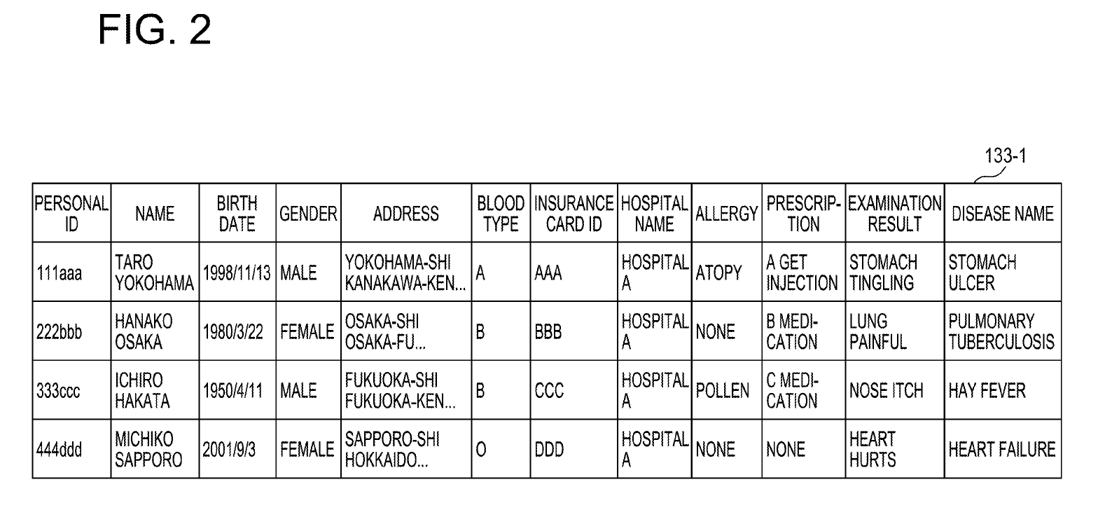

The user data DB 535 includes concealed electronic clinical charts. FIG. 2 is an example of a clinical chart information DB.

The clinical chart information DB 133-1 includes an electronic clinical chart of a patient of the hospital A. The clinical chart information DB 133-1 includes, as items, personal ID, name, birth date, gender, address, blood type, insurance card ID, hospital name, allergy, prescription, examination result, and disease name.

The personal ID is a unique identifier (identification information) for identifying a user, which is assigned to each user (patient).

The name, the birth date, the gender, the address, and the blood type are a patient's name, a patient's birth date, a patient's gender, a patient's address, and a patient's blood type, respectively. The insurance card ID is an ID given to an insured person by an insurer. The hospital name is the name of a hospital which examined a patient and input an electronic clinical chart of the patient.

The allergy is an allergy of a patient, the prescription is a prescription determined by consultation, the examination result is an examination result referred to at the time of consultation, and the disease name is a disease name determined by consultation.

The clinical chart information DB 133-2 has the same format as the clinical chart information DB 133-1.

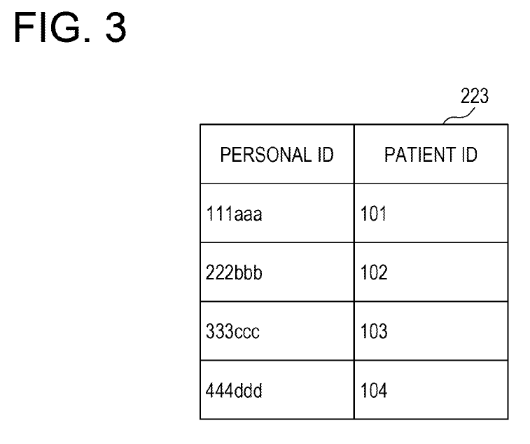

FIG. 3 is an example of a personal management DB. The personal management DB 223 includes, as items, personal ID and patient ID. In the personal management DB 223, the personal ID and the patient ID are recorded in association.

The personal ID is a unique identifier (identification information) for identifying a user, which is assigned to each user (patient). The personal ID is assigned to a user by, for example, an institution such as a government.

The patient ID is a unique identifier (identification information) for identifying a user, which is assigned to each user (patient). The patient ID is assigned to a user by the system management server 211.

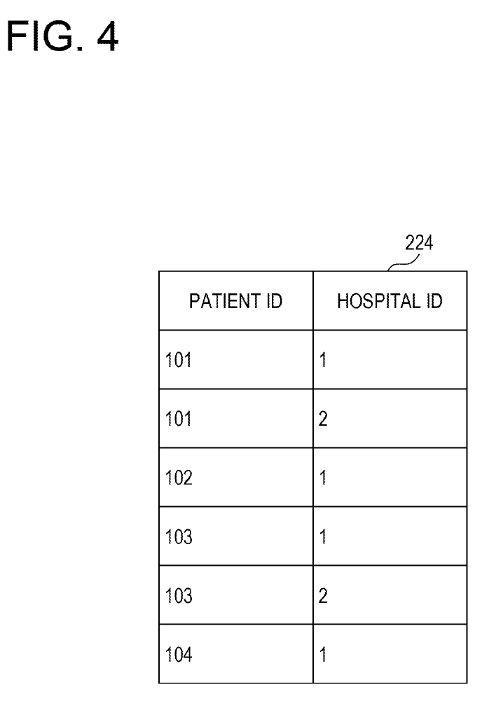

FIG. 4 is an example of a patient management DB. The patient management DB 224 includes, as items, patient ID and hospital ID. In the patient management DB 224, the patient ID, and the hospital ID are recorded in association.

The patient ID is a unique identifier (identification information) for identifying a user, which is assigned to each user (patient).

The hospital ID is a unique identifier (identification information) for identifying a hospital (hospital system), which is assigned to each hospital (hospital system). In one embodiment, a hospital ID=1 indicates a hospital A (hospital system 101-1), and a hospital ID=2 indicates a hospital B (hospital system 101-2).

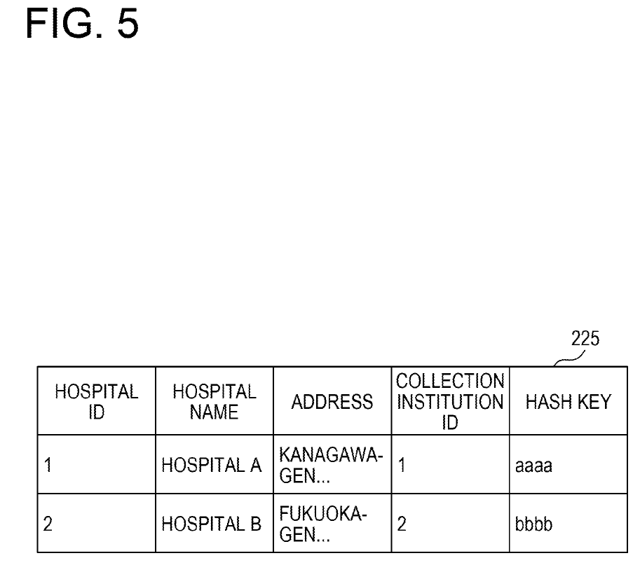

FIG. 5 is an example of a hospital management DB. The hospital management DB 225 includes information of the hospital system 101-i and information indicating the collection system 301-i of a transmission destination of an electronic clinical chart of the hospital system 101-i. The hospital management DB 225 includes, as items, hospital ID, hospital name, address, a collection institution ID, and a hash key. In hospital management DB 225, the hospital ID, the hospital name, the address, the collection institution ID, and the hash key are recorded in association.

The hospital ID is a unique identifier (identification information) for identifying a hospital (hospital system), which is assigned to each hospital (hospital system).

The hospital name is the name of a hospital. The address is the address of a hospital.

The collection institution ID is a unique identifier (identification information) for identifying a collection system 311-i. In one embodiment, a collection institution ID=1 indicates the collection system 301-1 and a collection institution ID=2 indicates the collection system 301-2. The collection institution ID indicates the collection system 311-i which is a transmission destination of an electronic clinical chart possessed by the hospital system 101-i.

The hash key is a hash key possessed by the hospital system 101-i. In FIG. 5, the hospital A (hospital system 101-1) has a hash key=aaaa. That is, a hash key 126-1=aaaa. Further, in FIG. 5, the hospital B (hospital system 101-2) has a hash key=bbbb. That is, a hash key 126-2=bbbb.

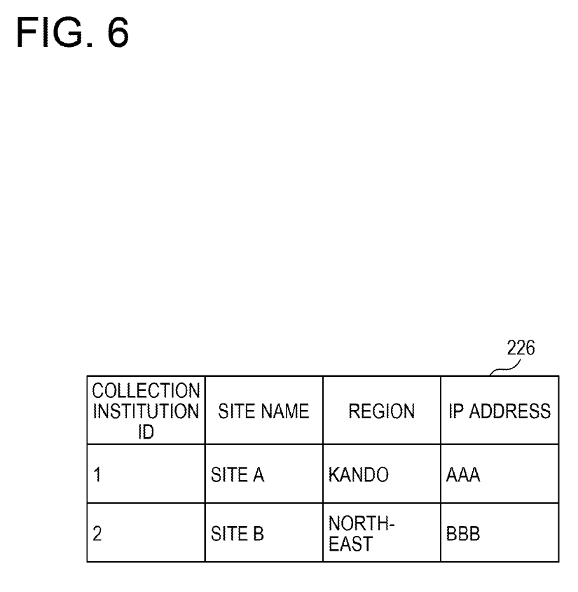

FIG. 6 is an example of a collection system management DB. The collection system management DB 226 includes information of the collection system 301-i. The collection system management DB 226 includes, as items, collection institution ID, site name, region, and IP address. In the collection system management DB 226, the collection institution ID, the site name, the region, and the IP address are recorded in association.

The collection institution ID is a unique identifier (identification information) for identifying the collection system 311-i.

The site name is the name of a site where the collection system 301-i is installed. The region indicates a region where the collection system 311-i is installed. The IP address is an IP address of the collection server 312-i.

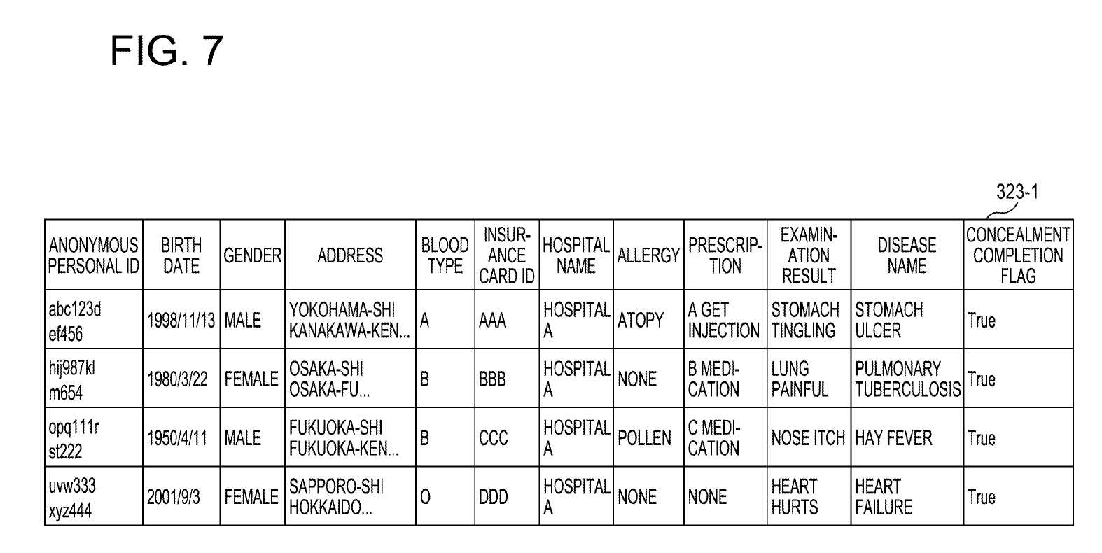

FIG. 7 is an example of an anonymous ID clinical chart information DB. The anonymous ID clinical chart information DB 323-1 includes, as items, anonymous personal ID, birth date, gender, address, blood type, insurance card ID, hospital name, allergy, prescription, examination result, disease name, and concealment flag. In the anonymous ID clinical chart information DB 323-1, the anonymous personal ID, the birth date, the gender, the address, the blood type, the insurance card ID, the hospital name, the allergy, the prescription, the examination result, the disease name, and the concealment flag are recorded in association.

The anonymous personal ID is a hash value obtained by hashing a personal ID with a hash key. For example, an anonymous personal ID=abc123def456 in the first row of the anonymous ID clinical chart information DB 323-1 in FIG. 7 is a hash value obtained by hashing a personal ID=111aaa in the first row of the clinical chart information DB 133-1 in FIG. 2 with the hash key 126-1. The anonymous personal ID is an example of an identifier.

The name, the birth date, the gender, the address, and the blood type are a patient's name, a patient's birth date, a patient's gender, a patient's address, and a patient's blood type, respectively. The insurance card ID is an ID given to an insured person by an insurer. The hospital name is the name of a hospital which examined a patient and input an electronic clinical chart of the patient.

The allergy is an allergy of a patient, the prescription is a prescription determined by consultation, the examination result is an examination result referred to at the time of consultation, and the disease names is a disease name determined by consultation.

The concealment flag indicates whether or not a concealment process has been performed on the corresponding data. “True” indicates that the concealment process has been completed, and “false” indicates that the concealment process has not been completed.

The anonymous ID clinical chart information DB 323-2 has the same format as the anonymous ID clinical chart information DB 323-1.

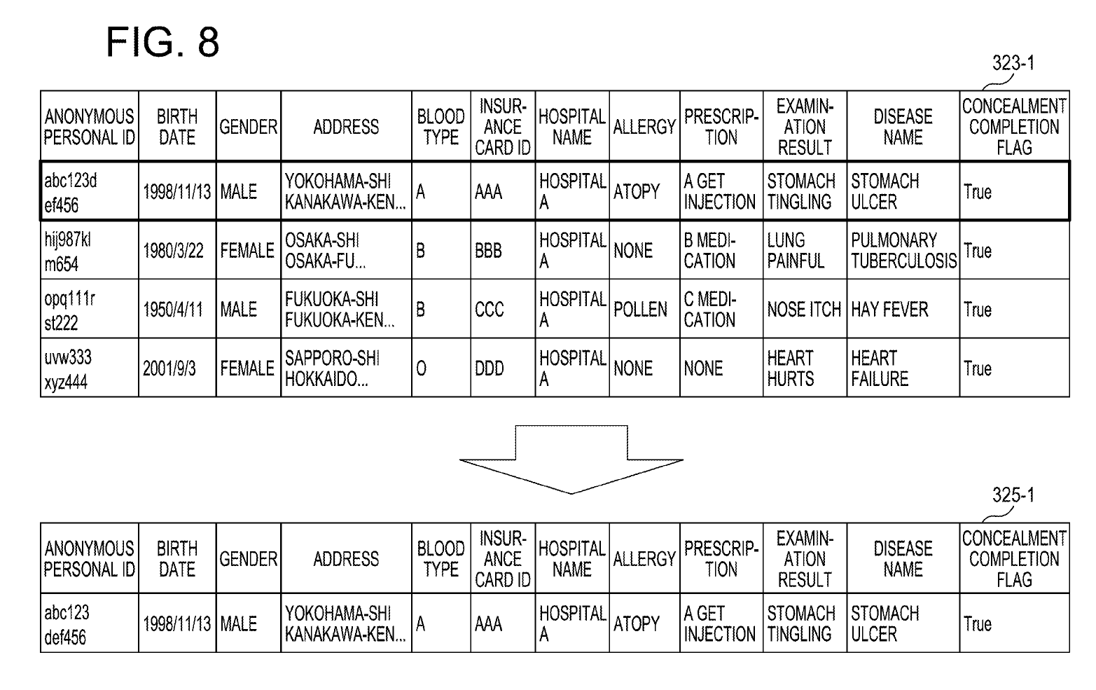

FIG. 8 illustrates an example of a temporary DB generated from the anonymous ID clinical chart information DB. The temporary DB 325-1 is generated when the collection system 301-1 transmits an electronic clinical chart included in the anonymous ID clinical chart information DB 323-1 based on a request from the service utilization system 401.

The temporary DB 325-1 has the same format as the anonymous ID clinical chart information DB 323-1. The temporary DB 325-2 has the same format as the anonymous ID clinical chart information DB 323-2.

In the anonymous ID clinical chart information DB 323-1 of FIG. 8, upon being requested for an electronic clinical chart with the anonymous personal ID=abc123def456, the transfer processing unit 315-1 extracts data on the first row of the anonymous ID clinical chart information DB 323-1 and writes the data in the temporary DB 325-1.

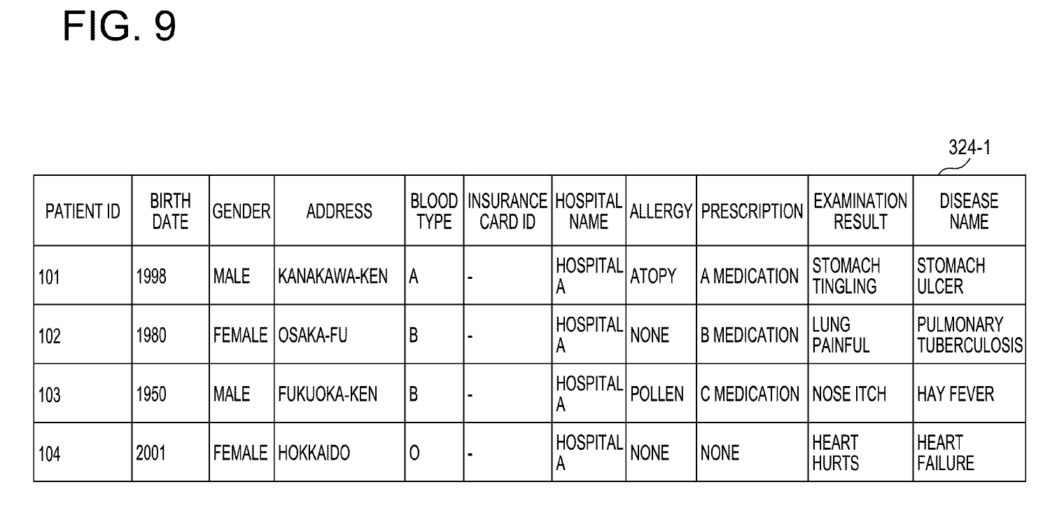

FIG. 9 is an example of a concealment clinical chart information DB. The concealment clinical chart information DB 324-1 is generated by using a concealment process for concealing data of a predetermined item of the anonymous ID clinical chart information DB 323-1. Further, in the concealment clinical chart information DB 324-1, a patient ID is used instead of the anonymous personal ID.

The concealment clinical chart information DB 324-1 includes, as items, patient ID, birth date, gender, address, blood type, insurance card ID, hospital name, allergy, prescription, examination result, and disease name. In one embodiment, among the items of the anonymous ID clinical chart information DB 323-1, data of the birth date, the address and the insurance card ID are concealed to generate the concealment clinical chart information DB 324-1.

The patient ID is a unique identifier (identification information) for identifying a user, which is assigned to each user (patient).

The birth date, the gender, the address, and the blood type are the age in a patient's birth date, a patient's gender, a prefecture in a patient's address, and a patient's blood type, respectively. Data of the insurance card ID is empty(−). The hospital name is the name of a hospital which examined a patient and input an electronic clinical chart of the patient.

The allergy is an allergy of a patient, the prescription is a prescription determined by consultation, the examination result is an examination result referred to at the time of consultation, and the disease name is a disease name determined by consultation.

In this way, in the concealment clinical chart information DB 324-1, the data of the birth date, address, and insurance card ID are simplified or deleted by the concealment process so that individuals are not specified.

The concealment clinical chart information DB 324-2 has the same format as the concealment clinical chart information DB 324-1.

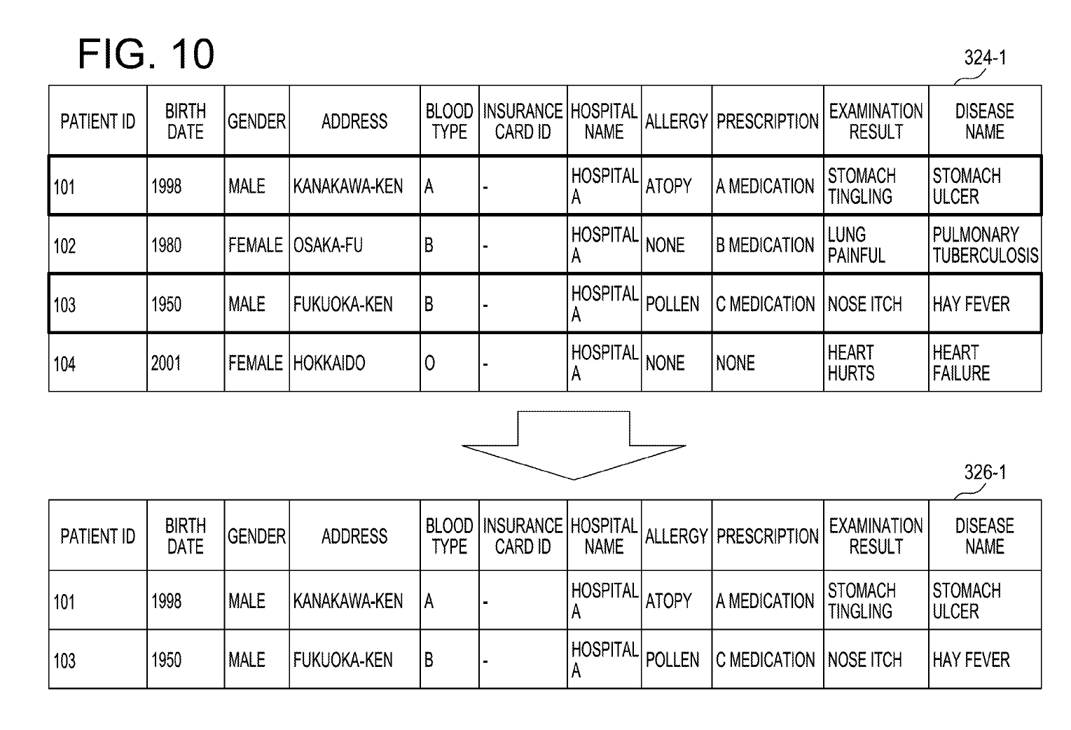

FIG. 10 is an example of a temporary DB generated from the concealment clinical chart information DB. The temporary DB 326-1 is generated when the collection system 301-1 transmits a concealed electronic clinical chart included in the concealment clinical chart information DB 324-1 based on a request from the research institution system 501.

The temporary DB 326-1 has the same format as the concealment clinical chart information DB 324-1. The temporary DB 326-2 has the same format as the concealment clinical chart information DB 324-2.

In the concealment clinical chart information DB 324-1 of FIG. 10, upon being requested for an electronic clinical chart of the gender=male, the transfer processing unit 315-1 extracts data of the first row and the third row of the concealment clinical chart information DB 324-1 and writes them in the temporary DB 326-1.

Next, the concealment process by the concealment unit 314-1 will be described. The concealment unit 314-1 conceals data of a predetermined item of the anonymous ID clinical chart information DB 323-1 and writes the concealed data in the concealment clinical chart information DB 324-1. The concealment unit 314-1 simplifies or deletes data of predetermined items so as not to specify an individual, and writes them in the concealment clinical chart information DB 324-1. Which item data to be concealed is preset. In one embodiment, the predetermined items are the birth date, the address and the insurance card ID, and the data of the birth date, the address and the insurance card ID are concealed.

For example, when concealing the birth date=1998/11/13 of the first row of the anonymous ID clinical chart information DB 323-1 in FIG. 7, the concealment unit 314-1 extracts the age in the birth date and writes it in the concealment clinical chart information DB 324-1. As a result, in FIG. 9, the birth date of the first row of the concealment clinical chart information DB 324-1 is “1998.”

When concealing the address=Yokohama-shi Kanagawa-ken of the first row of the anonymous ID clinical chart information DB 323-1 of FIG. 7, the concealment unit 314-1 extracts the prefecture out of the address and writes the prefecture in the concealment clinical chart information DB 324-1. As a result, in FIG. 9, the address of the first row of the concealment chart record information DB 324-1 is “Kanagawa-gen.”

When concealing the insurance card ID=AAA of the first row of the anonymous ID clinical chart information DB 323-1 of FIG. 7, the concealment unit 314-1 deletes data of the insurance card ID and writes empty(−) in the concealment clinical chart information DB 324-1. As a result, in FIG. 9, the insurance card ID of the first row of the concealment clinical chart information DB 324-1 is “empty(−).”

Further, the concealment unit 314-1 uses a patient ID in place of the anonymous personal ID in the concealment clinical chart information DB 324-1. Since different hospital systems 101-i have different hash keys 126-1, different hash values (anonymous personal IDs) are obtained for different hospital systems 101-i when the same personal ID is hashed. For this reason, with an anonymous personal ID, it is difficult to determine whether or not a plurality of electronic clinical charts is an electronic chart of the same person. Since the patient ID may be used to know that the plurality of electronic clinical charts is the electronic clinical chart of the same person, it is possible to merge the plurality of electronic clinical charts of the same person in the research institution system 501.

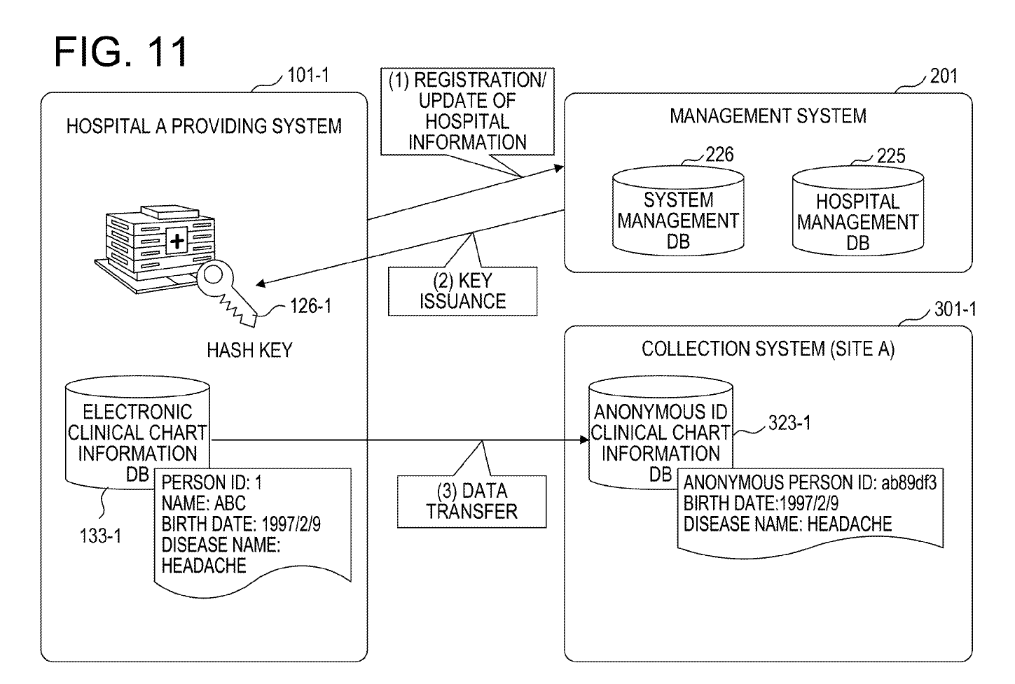

FIG. 11 is a view illustrating a data collection process according to an embodiment. In the information processing system 100, for example, data (electronic clinical charts) are collected from the hospital system 101-i into the collection system 301-i in the following sequence. Here, a case where electronic clinical charts are collected from the hospital system 101-1 into the collection system 301-1 as illustrated in FIG. 11 will be described. (1) The hospital system 101-1 requests the management system 201 to register or update the information of the hospital A. Upon receiving the request to register or update the information of the hospital A, the management system 201 registers the information of the hospital A in the hospital management DB 225 or updates the information of the hospital A. When the hospital system 101-1 does not have the hash key 126-1, it requests the management system 201 to issue the hash key 126-1. (2) The management system 201 generates the hash key 126-1 of the hospital A and transmits the hash key 126-1 of the hospital A to the hospital system 101-1. The hash key is a unique key that is different for each hospital. (3) The hospital system 101-1 uses the hash key 126-1 received from the management system 201 to generate an anonymous patient ID obtained by hashing a personal ID of an electronic clinical chart included in the electronic clinical chart information DB 133-1, and transmits the anonymous patient ID and data of the birth date, gender, address, blood type, insurance card ID, hospital name, allergy, prescription, examination result and disease name of the electronic clinical chart to the collection system 301. That is, the hospital system 101-1 transmits, as patient data, the anonymous patient ID and data other than the personal ID and the name of the electronic clinical chart to the collection system 301-1. The collection system 301-1 records the received anonymous patient ID and the electronic clinical chart data in the concealment clinical chart information DB 323-1.

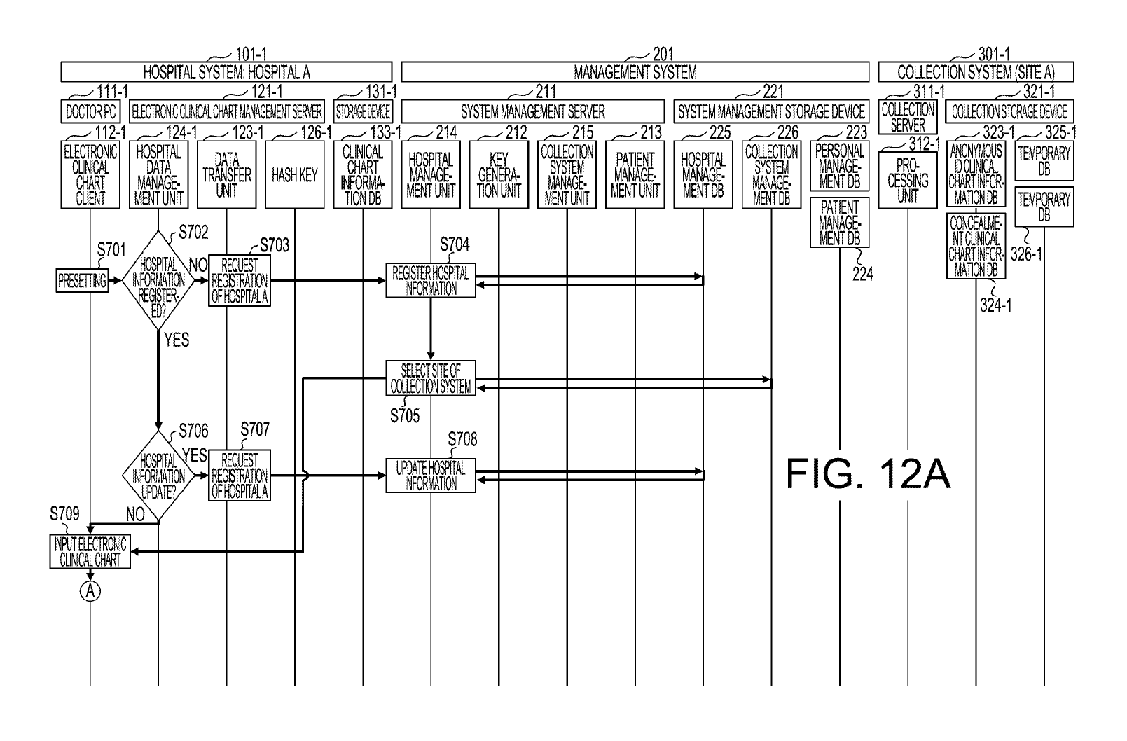

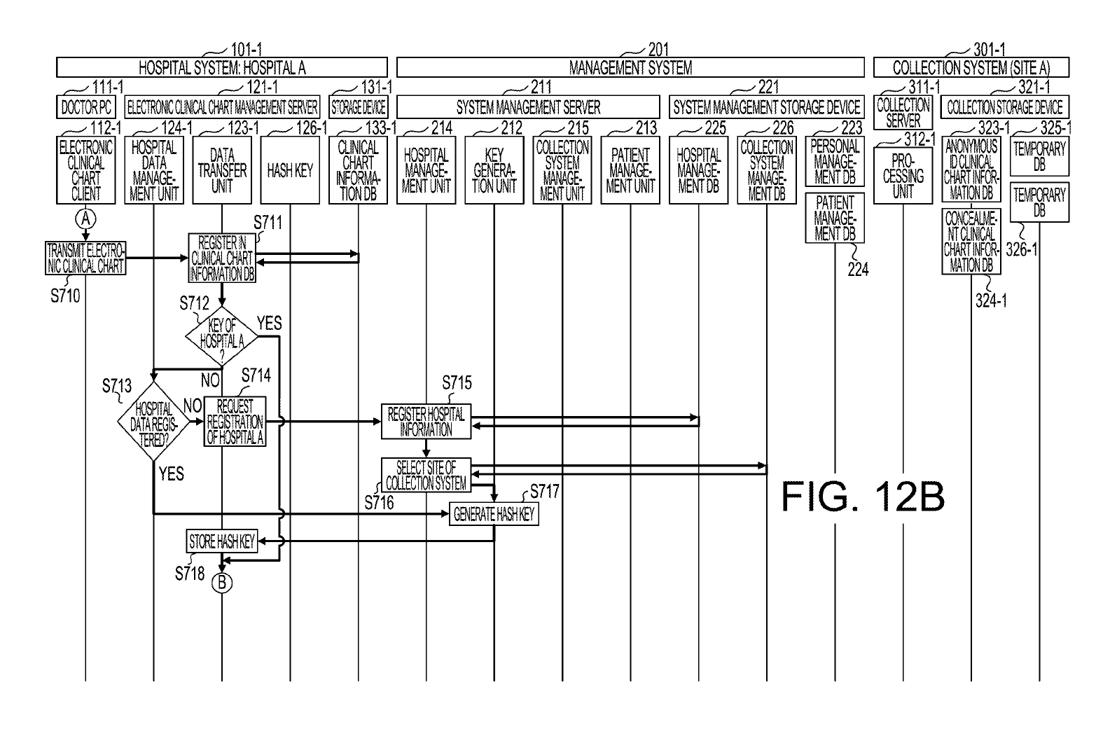

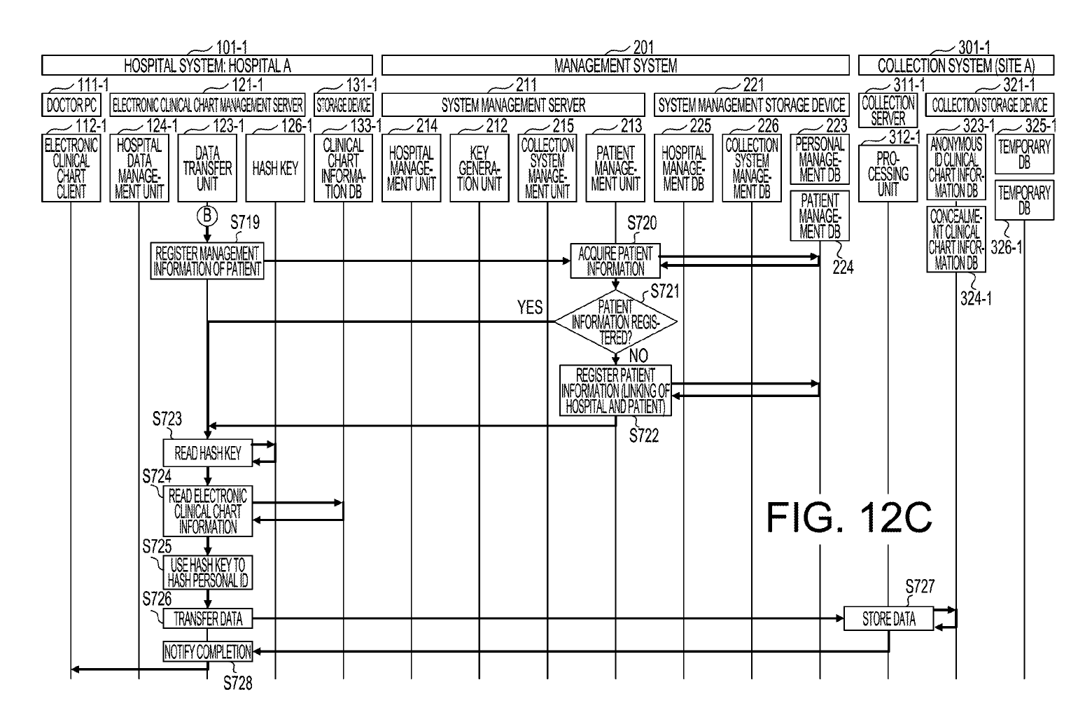

FIGS. 12A and 12C are sequence diagrams of a data collection process according to an embodiment. A case where electronic clinical charts are collected from the hospital system 101-1 into the collection system 301-1 will be described with reference to FIGS. 12A and 12C.

In step S701, the electronic clinical chart client 112-1 of the doctor PC 111-1 requests the electronic clinical chart management server 121-1 to perform presetting.

In step S702, when it is determined that the information (hospital information) of the hospital A has been registered, the control proceeds to step S706. When it is determined that the hospital information has not been registered, the control proceeds to step S703. When there is a case where the system management server 211 has been previously requested to register the hospital information, it is determined that the hospital information has been registered.

In the step S703, the data transfer unit 123-1 requests the system management server 211 to register the information of the hospital A.

In step S704, upon receiving the request from the data transfer unit 123-1, the hospital management unit 214 registers the information (hospital information) of the hospital A in the hospital management DB 225.

In step S705, the hospital management unit 214 refers to the collection system management DB 226 to select the collection system 301-i that collects the electronic clinical charts of the hospital A, from the collection system management DB 226. The hospital management unit 214 writes a collection institution ID indicating the selected collection system 301-i in the collection institution ID corresponding to the information of the hospital A of the hospital management DB 225.

In the step S706, when it is determined that there is an update of the information (hospital information) of the hospital A, the control proceeds to step S707. When it is determined that there is no update of the hospital information, the control proceeds to step S709.

In the step S707, the data transfer unit 123-1 requests the system management server 211 to register the information of the hospital A.

In step S708, upon receiving the request from the data transfer unit 123-1, the hospital management unit 214 updates the information (hospital information) of the hospital A of the hospital management DB 225.

In the step S709, the electronic clinical chart client 113-1 inputs patient data, examination information and the like to each item of the electronic clinical chart based on an operation by a doctor.

In step S710, the electronic clinical chart client 113-1 transmits the electronic clinical chart to which the data is input, to the electronic clinical chart management server 121-i.

In step S711, the data transfer unit 123-1 records the received electronic clinical chart in the clinical chart information DB 133-1.

In step S712, the data transfer unit 123-1 determines whether or not a hash key 133-1 is present in the storage unit 132-1. When it is determined that there is a hash key 133-1 in the storage unit 132-1, the control proceeds to step 719. When it is determined that there is no hash key 133-1 in the storage unit 132-1, the control proceeds to step 713.

In step S713, when it is determined that the information (hospital information) of the hospital A has been registered, the control proceeds to step S717. When it is determined that the hospital information has not been registered, the control proceeds to step S714. When there is a case where the system management server 211 has been previously requested to register the hospital information, it is determined that the hospital information has been registered.

In step S714, the data transfer unit 123-1 requests the system management server 211 to register the information of the hospital A.

In step S715, upon receiving the request from the data transfer unit 123-1, the hospital management unit 214 registers the information (hospital information) of the hospital A in the hospital management DB 225.

In step S716, the hospital management unit 214 refers to the collection system management DB 226 to select the collection system 301-i that collects the electronic clinical chart of the hospital A, from the collection system management DB 226. The hospital management unit 214 writes a collection institution ID indicating the selected collection system 301-i in the collection institution ID corresponding to the information of the hospital A of the hospital management DB 225.

In step S717, the key generation unit 212 generates the hash key 126-1 of the hospital A and transmits the hash key 126-1 of the hospital A to the electronic clinical chart management server 121-1.

In step S718, the data transfer unit 123-1 receives the hash key 126-1 and stores it in the storage unit 125-1.

In step S719, the data transfer unit 123-1 transmits a request to register the management information of a patient to the system management server 211. The registration request includes a patient's personal ID.

In step S720, the patient management unit 213 receives the registration request and refers to the personal management DB 223 and the patient management DB 224 to acquire patient information (patient ID corresponding to the personal ID included in the registration request and hospital ID corresponding to the patient ID).

In step S721, when it is determined that the patient information has already been registered (that is, when the patient information could be acquired), the control proceeds to step S723. When it is determined that the patient information has not been registered (that is, when the patient information could not be acquired), the control proceeds to step S722.

In step S722, the patient management unit 213 assigns a patient ID to the personal ID included in the registration request and records the personal ID and the patient ID in the personal management DB 223. In addition, the patient management unit 213 records the patient ID corresponding to the personal ID included in the registration request and the hospital ID of the hospital A in the patient management DB 224 (linking the hospital ID and the patient ID).

In step S723, the data transfer unit 123-1 reads the hash key 126-1 from the storage unit 125-1.

In step S724, the data transfer unit 123-1 reads the electronic clinical chart included in the clinical chart information DB 133-1.

In step S725, the data transfer unit 123-1 uses the hash key 126-1 to hash the personal ID of the read electronic clinical chart, and calculates an anonymous personal ID which is a hash value.

In step S726, the data transfer unit 123-1 transmits the anonymous personal ID instead of the personal ID, and data of items other than the read personal ID of the read electronic clinical chart, to the collection server 311-1.

In step S727, the collection unit 313-1 records the anonymous personal ID and the data of items other than the personal medical ID of the electronic clinical chart in the anonymous ID clinical information DB 323-1.

In step S728, the data transfer unit 123-1 transmits a completion notification to the doctor PC 111-1.

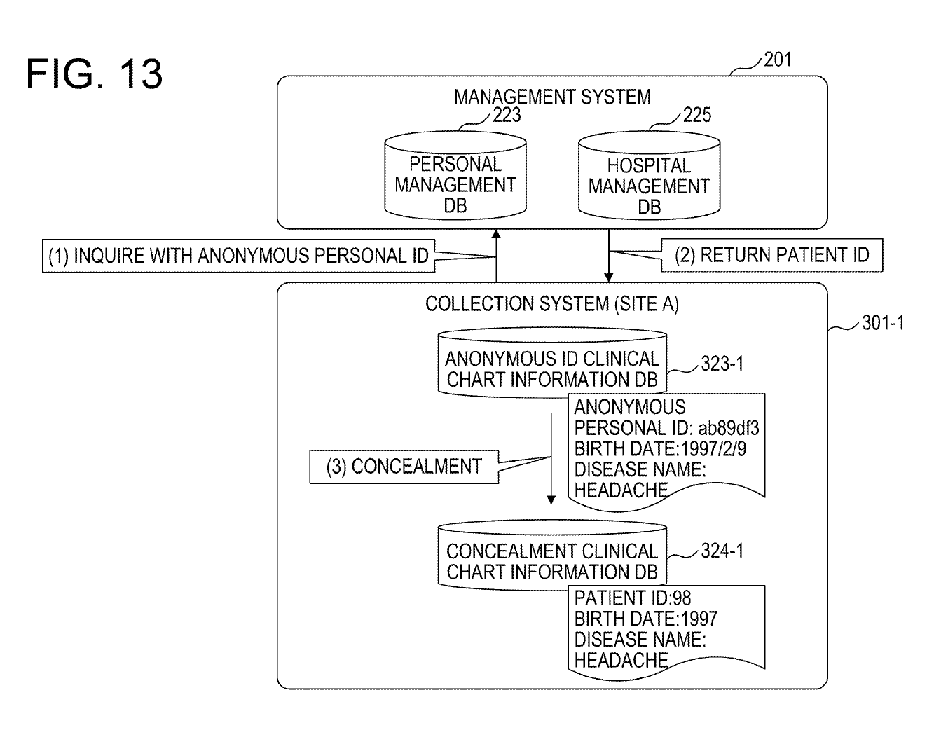

FIG. 13 is view illustrating a concealment process according to an embodiment. In the information processing system 100, for example, a concealment process of a non-concealed electronic clinical chart included in the anonymous ID clinical chart information DB 323-1 is performed in the following sequence.

In FIG. 13, it is assumed that an electronic clinical chart including anonymous personal ID=ab89df3, birth date=1997/2/9, and disease name=headache is recorded in the anonymous ID clinical chart information DB 323-1. A case where the collection system 301-1 conceals data of a predetermined item of the electronic clinical chart including the anonymous personal ID=ab89df3 will be described below. Here, the predetermined item to be subjected to the concealment process is the birth date. (1) The collection system 301-1 inquires the management system 202 about a patient ID corresponding to the anonymous personal ID=ab89df3. (2) Based on the personal management DB 223 and the hospital management DB 225, the management system 201 finds the patient ID corresponding to the personal ID corresponding to the anonymous personal ID=ab89df3. Here, the patient ID corresponding to the personal ID corresponding to the anonymous personal ID=ab89df3 is “98.” The management system 201 returns (transmits) the patient ID=98 to the collection system 301-1. (3) The collection system 301-1 writes a patient ID instead of the anonymous personal ID in the concealment clinical chart information DB 324-1. The collection system 301-1 simplifies the data of birth date by performing the concealment process on birth date=1997/2/9 and stores the concealed birth date in the concealment clinical chart information DB 324-1. Through the concealment process, the concealed birth date is 1997. The collection system 301-1 writes non-concealed disease name data in the concealment clinical chart information DB 324-1.

That is, the collection system 301-1 writes the patient ID=98, the concealed birth date=1997 and the disease name=headache in the concealment clinical chart information DB 324-1.

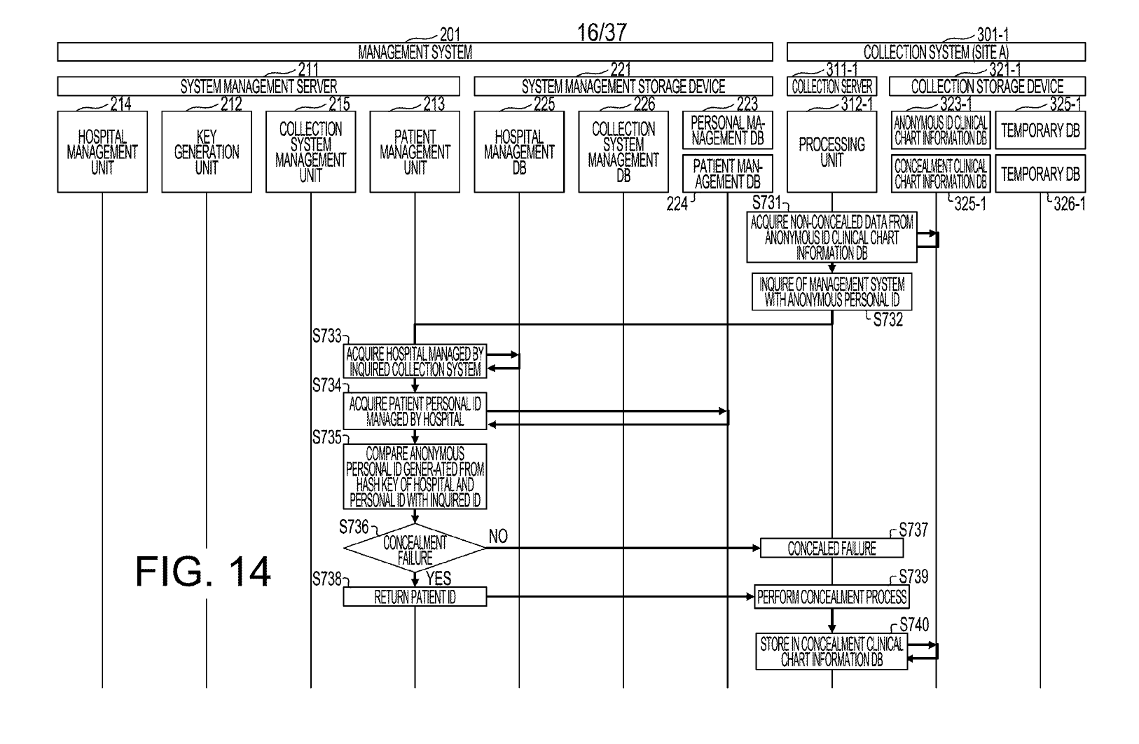

FIG. 14 is a sequence diagram of a concealment process according to an embodiment. A case where a concealment process is performed in the collection system 301-1 will be described below with reference to FIG. 14.

In step S731, the concealment unit 314-1 reads a non-concealed electronic clinical chart from the anonymous ID clinical chart information DB 323-1. A record with a concealment complement flag=false corresponds to the non-concealed electronic clinical chart. Here, it is assumed that an electronic clinical chart of one person (corresponding to one row of the anonymous ID clinical chart information DB 323-1) is read out.

In step S732, the concealment unit 314-1 transmits an inquiry including an anonymous patient ID included in the read electronic clinical chart to the system management server 211.

In step S733, the patient management unit 213 receives the inquiry and refers to the hospital management DB 325 to acquire hospital information (hospital ID and hash key) managed by the inquiry source collection system 301-1. The collection institution ID of the collection system 301-1 is 1. In the hospital management DB 325 of FIG. 5, the hospital ID corresponding to the collection institution ID=1 is 1 and a hash key corresponding to the hospital ID=1 (i.e., the hash key 126-1 of the hospital A) is aaaa.

In step S734, the patient management unit 213 refers to the patient management DB 324 to acquire a patient ID corresponding to the acquired hospital ID=1. The patient management unit 213 refers to the personal management DB 223 to acquire a personal ID corresponding to the acquired patient ID. Here, it is assumed that a plurality of personal IDs is acquired.

In step S735, the patient management unit 213 uses the hash key (=aaaa) corresponding to the acquired hospital ID=1 to hash each of the acquired personal IDs, and generates a plurality of anonymous personal IDs corresponding to the plurality of acquired personal IDs which are hash values. The patient management unit 213 compares the anonymous personal ID included in the inquiry with the generated plural anonymous personal IDs. The patient management unit 213 stores, in the system management server 211, the correspondence relationship between the acquired personal IDs and the anonymous personal IDs generated from the acquired personal IDs.

In step S736, the patient management unit 213 determines whether or not an anonymous personal ID matching the anonymous personal ID included in the inquiry is one of the plurality of generated anonymous personal IDs. When it is determined that the anonymous personal ID matching the anonymous personal ID included in the inquiry is one of the generated plural anonymous personal IDs, the control proceeds to step S738. When it is determined that the anonymous personal ID matching the anonymous personal ID included in the inquiry is not one of the generated plural anonymous personal IDs, the patient management unit 213 notifies the collection server 311-1 that there is no patient ID corresponding to the inquired anonymous personal ID, and then the control proceeds to step S737.

In step S737, since there is no patient ID corresponding to the anonymous personal ID, the patient management unit 213 ends the process (unsuccessful concealment).

In step S738, the patient management unit 213 returns (transmits) the patient ID corresponding to the personal ID used to generate the anonymous personal ID matching the anonymous personal ID included in the inquiry to the collection server 311-1.

In step S739, the concealment unit 314-1 receives the patient ID. The concealment unit 314-1 performs the concealment process on the data of the predetermined items of the electronic clinical chart read from the anonymous ID clinical chart information DB 323-1. In one embodiment, the predetermined items are the birth date, the address and the insurance card ID.

In step S740, the concealment unit 314-1 registers the patient ID, the concealed data of the predetermined items, and the data of items (gender, blood type, hospital name, allergy, prescription, examination result, and disease name) except for the anonymous patient ID other than the predetermined items, in the concealment clinical chart information DB 324-1. As a result, the concealment clinical chart information DB 324-1 including the concealed electronic clinical chart as illustrated in FIG. 9 is generated.

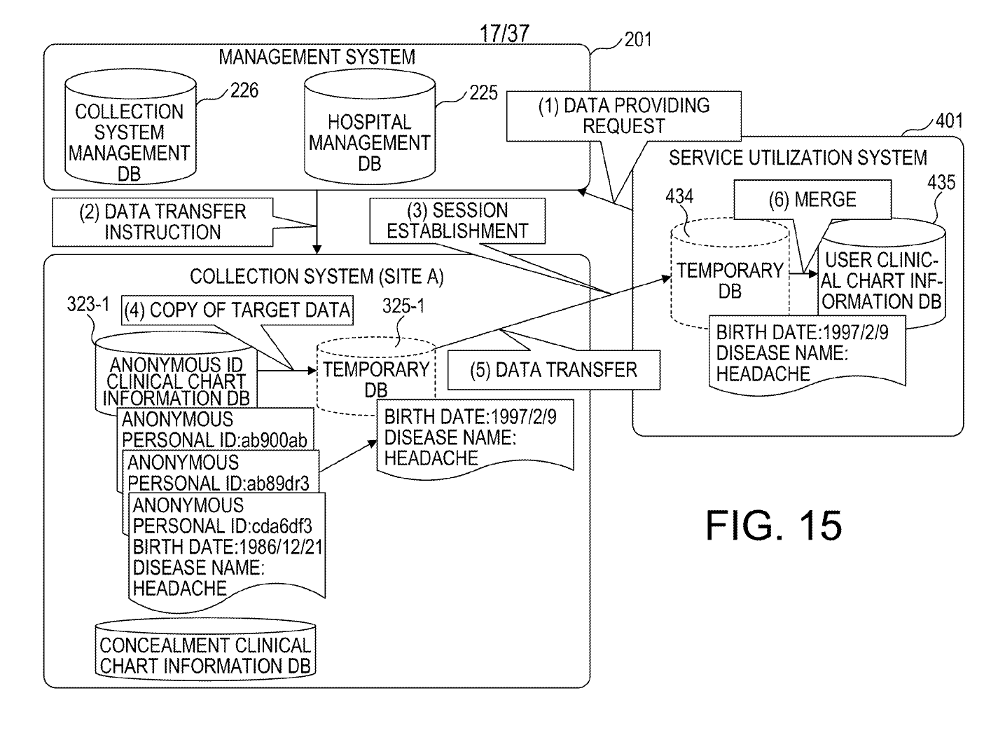

FIG. 15 is a view illustrating a process of transmitting non-concealed data to a service utilization system according to an embodiment.

In the information processing system 100, for example, a non-concealed electronic clinical chart included in the anonymous ID clinical chart information DB 323-i is transmitted to the service utilization system 401 in the following sequence. (1) The service utilization system 401 requests the management system 201 to provide data of a user under the consent of the user. At this time, the request to the data includes a personal ID of the user. (2) Upon receiving the request from the service utilization system 401, the management system 201 calculates an anonymous patient ID corresponding to the personal ID included in the request and instructs each collection system 301-I to transfer the data including the calculated anonymous patient ID. FIG. 15 illustrates a case where the management system 201 instructs the collection system 301-1 to transfer data. In FIG. 15, an anonymous patient ID=ab89dr3 is calculated. (3) The collection system 301-1 instructed to transfer the data establishes a session for data transfer with the data management server 421 of the service utilization system 401 of the transfer destination. (4) The collection system 301-1 extracts an electronic clinical chart corresponding to the anonymous patient ID received from the management system 201 from the anonymous ID clinical chart information DB 323-1 and copies the electronic clinical chart into the temporary DB 325-1. At this time, since an anonymous patient ID being managed by the collection system 301-1 may not be notified to the service utilization system 401, it is excluded from the electronic clinical chart to be copied into the temporary DB 325-1. In FIG. 15, an electronic clinical chart corresponding to the anonymous patient ID=ab89dr3 is extracted and copied into the temporary DB 325-1. (5) After copying the electronic clinical chart as a data transfer target into the temporary DB 325-1, the collection system 301-1 transmits the temporary DB 325-1 to the service utilization system 401. (6) The service utilization system 401 stores the received temporary DB 325-1 as the temporary DB 434, merges the temporary DB 434 into the user clinical chart information DB 435, and deletes the temporary DB 434.

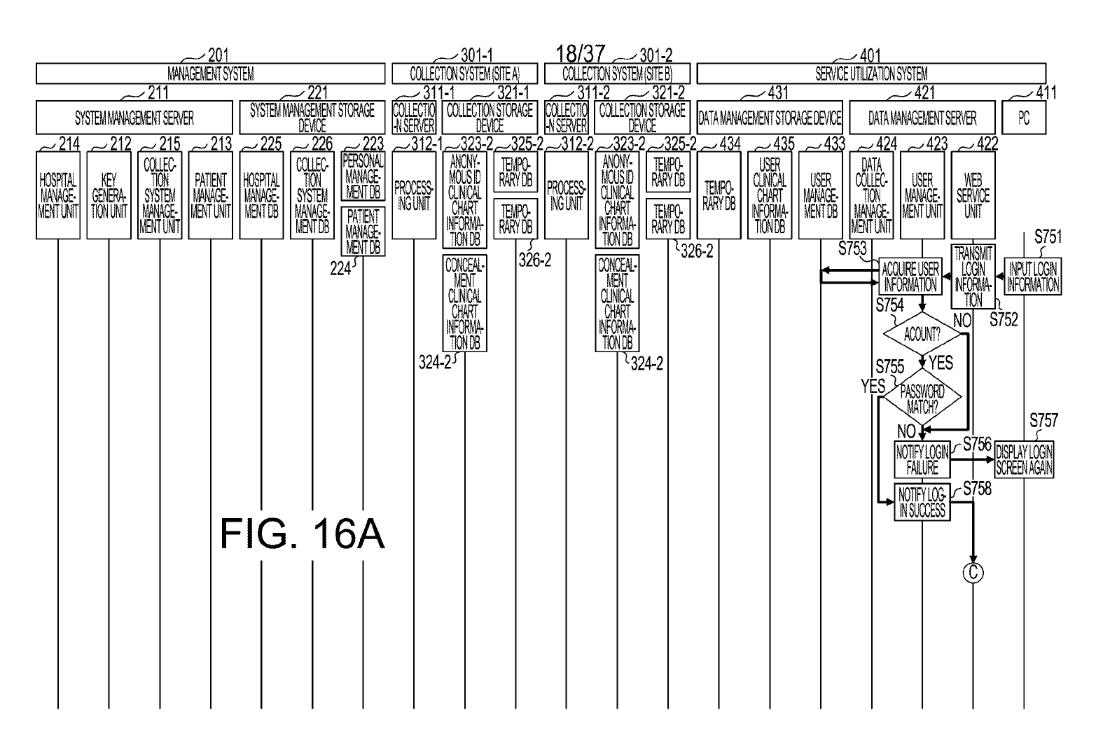

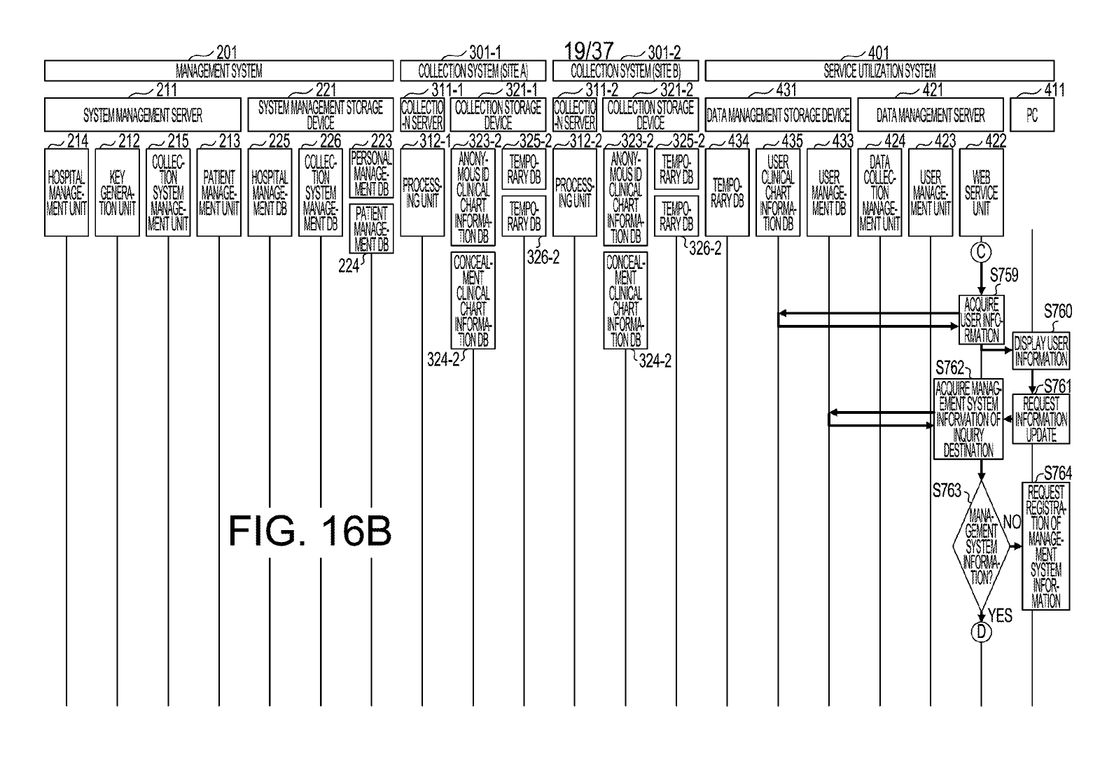

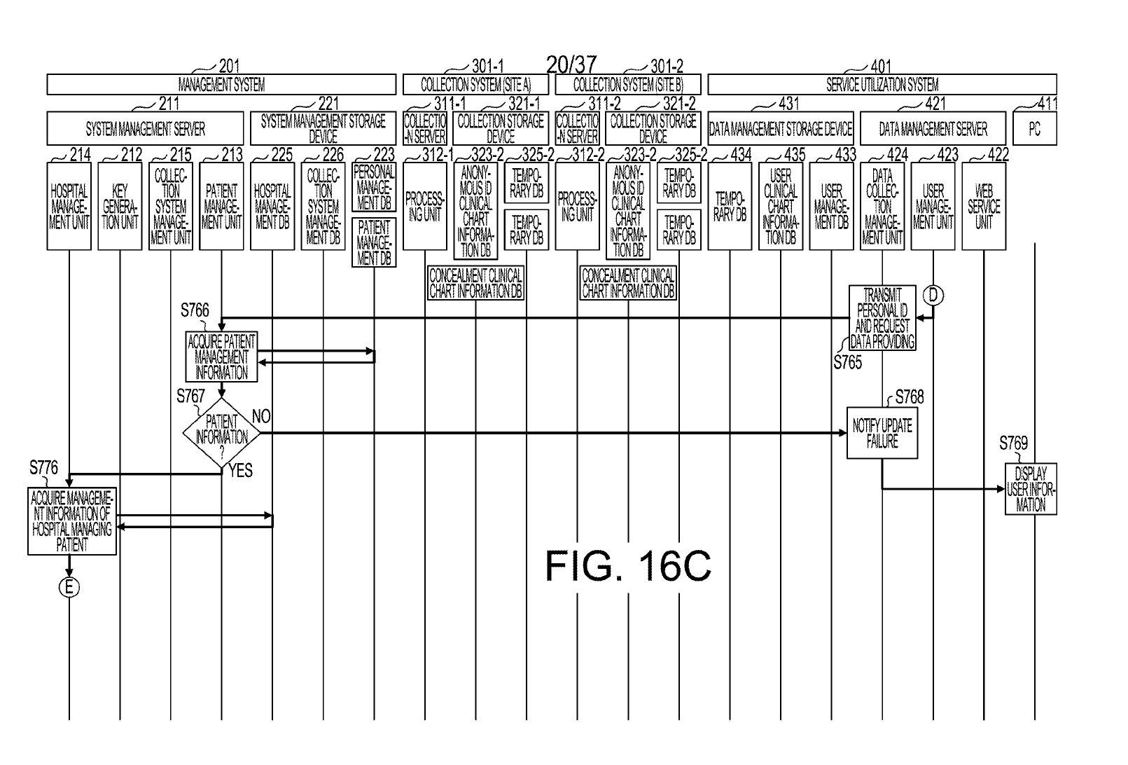

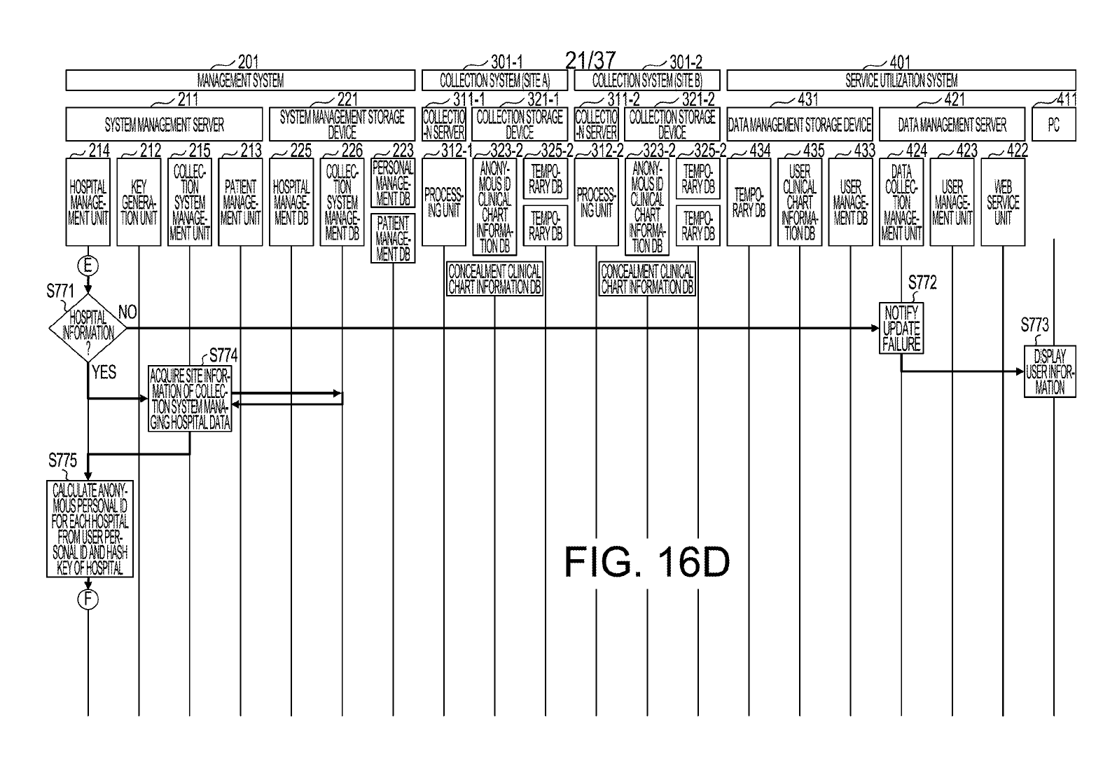

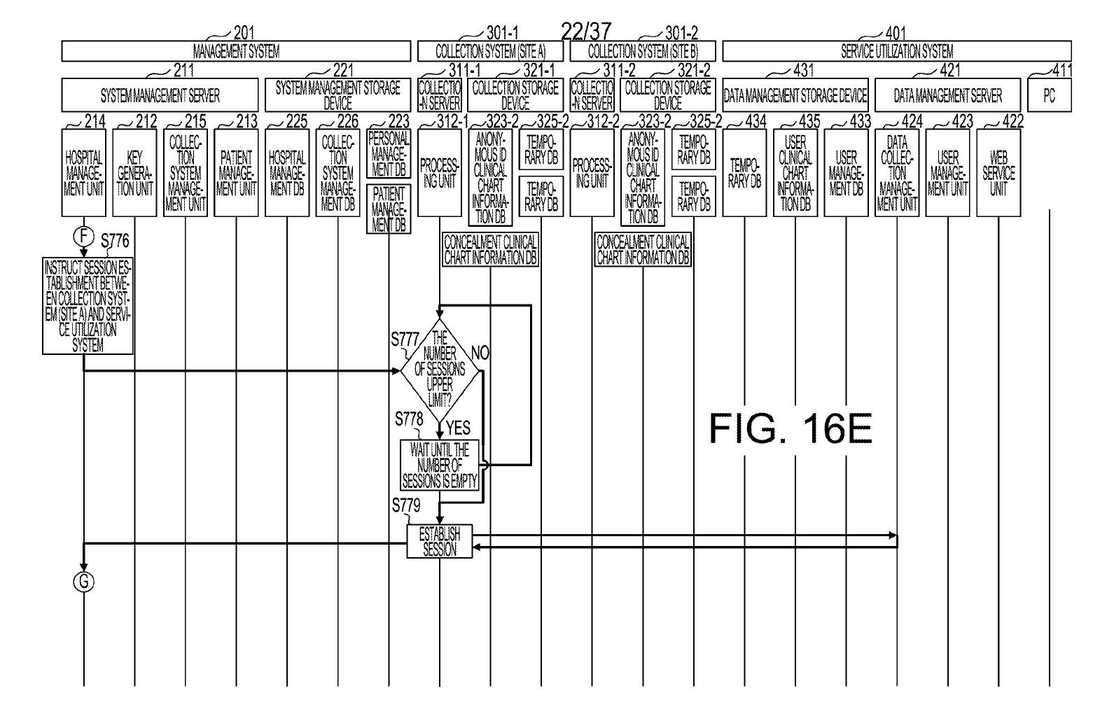

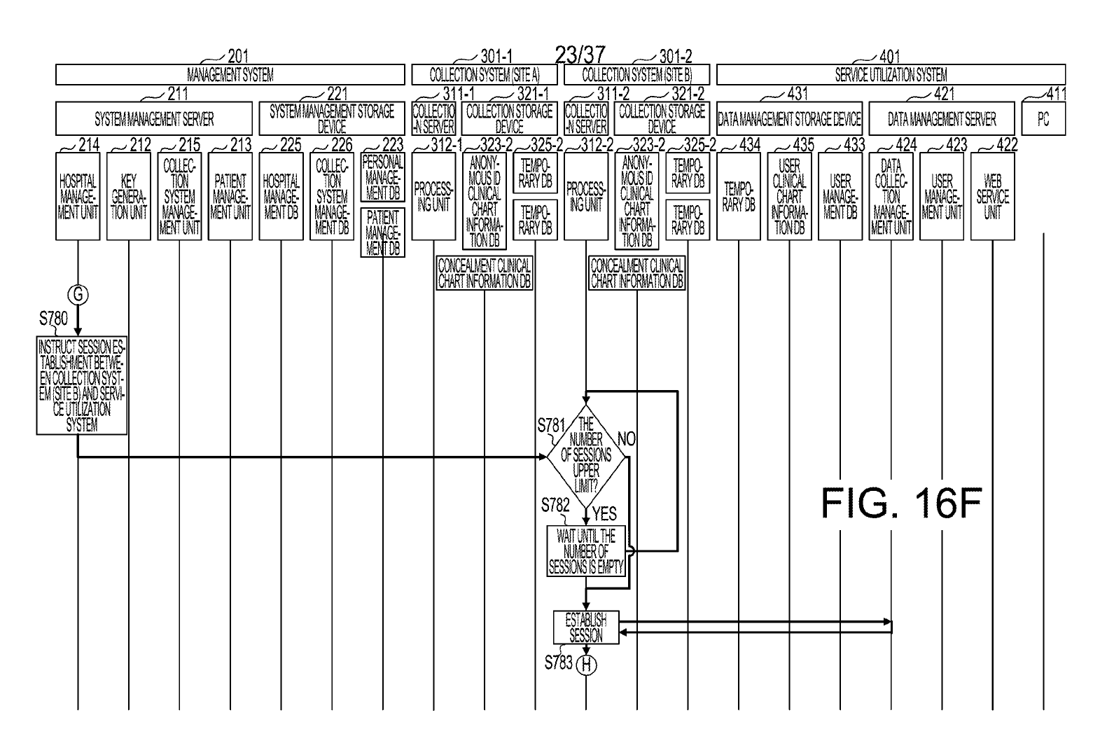

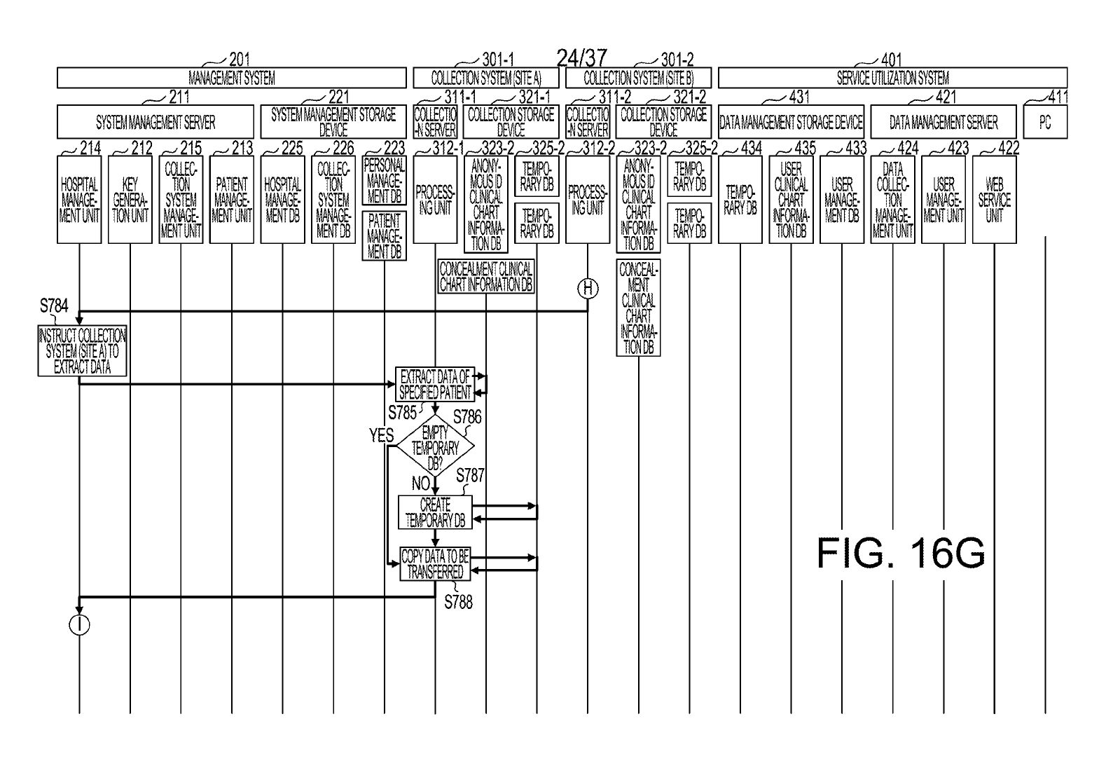

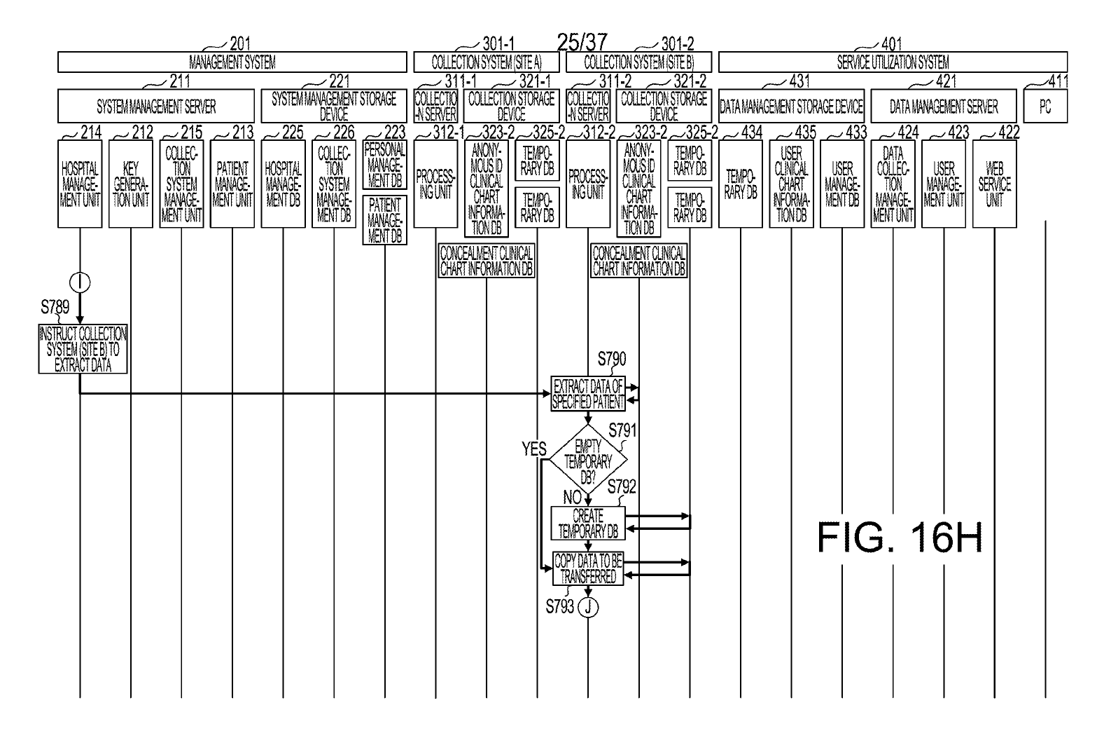

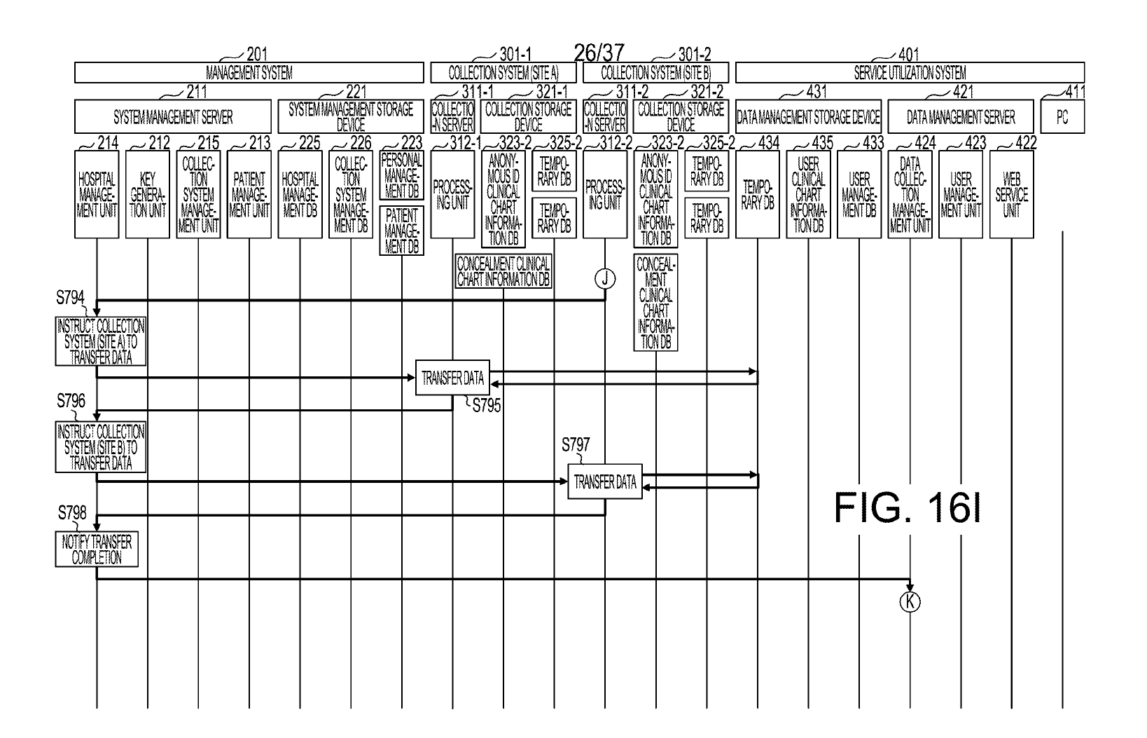

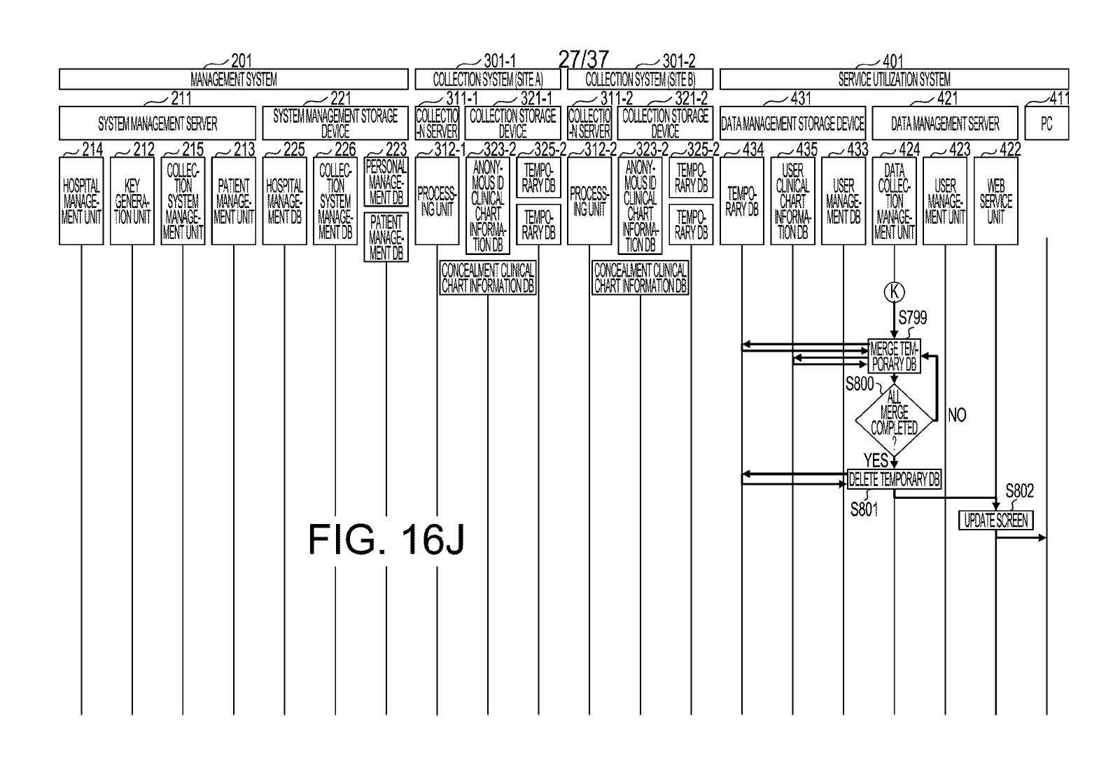

FIGS. 16A to 16J are sequence diagrams of a process of transmitting non-concealed data to a service utilization system according to an embodiment.

In step S751, the PC 411 uses a browser, a GUI or the like to input login information (account and password) to the data management server 211 based on an operation by a user.

In step S752, the web service unit 422 transmits the login information to the user management unit 424.

In step S753, the user management unit 424 receives the login information and acquires user information from the user management DB 433. The user information includes an account, a password, and a personal ID.

In step S754, the user management unit 424 determines whether or not the account included in the login information exists in the acquired user information. When it is determined that the account included in the login information exists in the acquired user information, the control proceeds to step S755. When it is determined that the account included in the login information does not exist in the acquired user information, the control proceeds to step S756.

In step S755, the user management unit 424 determines whether a password corresponding to the account included in the acquired user information matches the password included in the login information. When it is determined that the password corresponding to the account included in the acquired user information matches the password included in the login information, the control proceeds to step S758. When it is determined that they do not match each other, the control proceeds to step S756.

In step S756, the user management unit 424 notifies the PC 411 of the login failure.

In step S757, the PC 411 displays the login failure on a display unit (not illustrated) and also displays an input screen of login information on the display unit (not illustrated).

In step S758, the user management unit 424 notifies the web service unit 422 of the login success.

In step S759, the web service unit 422 acquires an electronic clinical chart of the user from the user clinical chart information DB 435 and transmits the electronic clinical chart to the PC 411.

In step S760, the PC 411 displays the received electronic clinical chart of the user on the display unit (not illustrated).

In step S761, the PC 411 requests the data management server 421 to update the information.

In step S762, upon receiving the request, the web service unit 422 refers to the user management DB 433 to acquire the information of the management system 201.

In step S763, when it is determined that the information of the management system 201 does not exist (the information of the management system 201 is not acquired from the user management DB 433), the control proceeds to step S764. When it is determined that there exists the information of the management system 201 (the information of the management system 201 is acquired from the user management DB 433), the control proceeds to step S765.

In step S764, the PC 411 requests the management system 201 to register the information, and the process is ended.

In step S765, the data collection management unit 424 transmits a request to provide data including a personal ID corresponding to the user's account to the system management server 211. That is, the data collection management unit 424 requests an electronic clinical chart associated with the personal ID.

In step S766, the patient management unit 213 refers to the personal management DB 223 and the patient management DB 224 to acquire management information of the user (patient) (a patient ID corresponding to the personal ID included in the data providing request and a hospital ID corresponding to the patient ID).

In step S767, when it is determined that there exists no patient management information (when the personal ID included in the data providing request is not present in the personal management DB 223), the patient management unit 213 transmits an update failure to the data management server 421, and the control proceeds to S768. When it is determined that there exists patient management information, the control proceeds to step S770.

In step S768, the data collection management unit 424 transmits the update failure to the PC 411.

In step S768, the PC 411 displays an update failure notification and the electronic clinical chart of the user on the display unit (not illustrated).

In step S770, the hospital management unit 214 refers to the hospital management DB 225 to acquire information of a hospital managing the patient's electronic clinical chart (including a hash key possessed by the hospital system of the hospital). That is, the hospital management unit 214 refers to the hospital management DB 225 to acquire information corresponding to the hospital ID corresponding to the patient ID of the patient.

In step S771, when it is determined that there exists no information of a hospital managing the patient's electronic clinical chart (when the hospital ID corresponding to the patient's patient ID is not present in the hospital management DB 225), the patient management unit 213 transmits an update failure to the data management server 421, and the control proceeds to step S772. When it is determined that there exists information of the hospital, the control proceeds to step S774.

In step S772, the data collection management unit 424 transmits the update failure to the PC 411.

In step S773, the PC 411 displays an update failure notification and the electronic clinical chart of the user on the display unit (not illustrated).

In step S774, the collection system management unit 215 refers to the collection system management DB 226 to acquire information of the collection system 301-i managing the electronic clinical chart transmitted from the hospital managing the patient's electronic clinical chart. That is, the collection system management unit 215 refers to the collection system management DB 226 to acquire information corresponding to the collection institution ID corresponding to the hospital ID corresponding to the patient ID of the patient.

In step S775, the hospital management unit 214 uses the hash key 126-i of the hospital managing the patient's electronic clinical chart to hash the personal ID of the user, and calculates an anonymous personal ID of the user for each hospital managing the patient's electronic clinical chart.

Here, it is assumed that a user is consulted at both the hospital A and the hospital B and the user's electronic clinical chart exists in each of the hospital system 101-1 and the hospital system 101-2. It is also assumed that the electronic clinical chart of the hospital system 101-1 of the hospital A is collected (managed) by the collection system 301-1 of the site A and the electronic clinical chart of the hospital system 101-2 of the hospital B is collected (managed) by the collection system 301-2 of the site B.

In step S776, the hospital management unit 214 instructs the collection server 311-1 of the collection system 301-1 to establish a session for data transfer between the collection server 311-1 and the data management server 421 of the service utilization system 401.

In step S777, when it is determined that the current number of sessions of the collection server 311-1 exceeds an upper limit, the control proceeds to step S778. When it is determined that the current number of sessions of the collection server 311-1 does not exceed the upper limit, the control proceeds to S779.

In step S778, the transfer processing unit 315-1 waits until the current number of sessions of the collection server 311-1 falls below the upper limit. When the current number of sessions falls below the upper limit, the control proceeds to step S777.

In step S779, the transfer processing unit 315-1 establishes a session with the data management server 421 of the service utilization system 401.

In step S780, the hospital management unit 214 instructs the collection server 311-2 of the collection system 301-2 to establish a session for data transfer between the collection server 311-2 and the data management server 421 of the service utilization system 40.

In step S781, when it is determined that the current number of sessions of the collection server 311-2 exceeds an upper limit, the control proceeds to step S782. When it is determined that the current number of sessions of the collection server 311-2 does not exceed the upper limit, the control proceeds to S783.

In step S782, the transfer processing unit 315-2 waits until the current number of sessions of the collection server 311-2 falls below the upper limit. When the current number of sessions falls below the upper limit, the control proceeds to step S781.

In step S783, the transfer processing unit 315-2 establishes a session with the data management server 421 of the service utilization system 401.

In step S784, the hospital management unit 214 transmits an instruction (request) for data extraction to the collection server 311-1 of the collection system 301-1. The instruction includes an anonymous personal ID calculated from the personal ID of the user using the hash key 126-1 of the hospital system 101-1 of the hospital A collected by the collection system 301-1.

In step S785, upon receiving the instruction, the transfer processing unit 315-1 extracts the electronic clinical chart of the user corresponding to the anonymous personal ID included in the instruction from the anonymous ID clinical chart information DB 323-1.

In step S786, when it is determined that the empty temporary DB 325-1 is present in the storage unit 322-1, the control proceeds to step S788. When it is determined that the empty temporary DB 325-1 is not present in the storage unit 322-1, the control proceeds to step S787.

In step S787, the transfer processing unit 315-1 creates the empty temporary DB 325-1.

In step S788, the transfer processing unit 315-1 copies the extracted electronic clinical chart into the temporary DB 325-1.

In step S789, the hospital management unit 214 transmits an instruction (request) for data extraction to the collection server 311-2 of the collection system 301-2. The instruction includes an anonymous personal ID calculated from the personal ID of the user using the hash key 126-2 of the hospital system 101-2 of the hospital B collected by the collection system 301-2.

In step S790, upon receiving the instruction, the transfer processing unit 315-2 extracts the electronic clinical chart of the user corresponding to the anonymous personal ID included in the instruction from the anonymous ID clinical chart information DB 323-2.

In step S791, when it is determined that the empty temporary DB 325-2 is present in the storage unit 322-2, the control proceeds to step S793. When it is determined that the empty temporary DB 325-2 is not present in the storage unit 322-2, the control proceeds to step S792.

In step S792, the transfer processing unit 315-2 creates the empty temporary DB 325-2.

In step S793, the transfer processing unit 315-2 copies the extracted electronic clinical chart into the temporary DB 325-1.

In step S794, the hospital management unit 214 transmits an instruction of data transfer to the collection server 311-1 of the collection system 301-1.

In step S795, the transfer processing unit 315-1 transmits the temporary DB 325-1 to the data management server 421, and the data collection management unit 424 stores the received temporary DB 325-1 as the temporary DB 434-1 in the storage unit 432.

In step S796, the hospital management unit 214 transmits an instruction of data transfer to the collection server 311-2 of the collection system 301-2.

In step S797, the transfer processing unit 315-2 transmits the temporary DB 325-2 to the data management server 421, and the data collection management unit 424 stores the received temporary DB 325-2 as the temporary DB 432-2 in the storage unit 432.

In step S798, the hospital management unit 214 notifies the data management server 421 of the completion of the transfer.

In step S799, the data collection management unit 424 merges an unmerged temporary DB 325-i into the user clinical chart information 435.

In step S800, when it is determined that all temporary DBs 325-i have been merged into the user clinical chart information 435, the control proceeds to step S801. When it is determined that there is an unmerged temporary DB 325-i, the control returns to step S799.

In step S801, the data collection management unit 424 deletes the temporary DB 325-i.

In step S802, the web service unit 422 acquires the updated electronic clinical chart of the user from the user clinical chart information DB 435 and transmits the electronic clinical chart to the PC 411. The PC 411 displays the updated electronic clinical chart on the display unit (not shown).

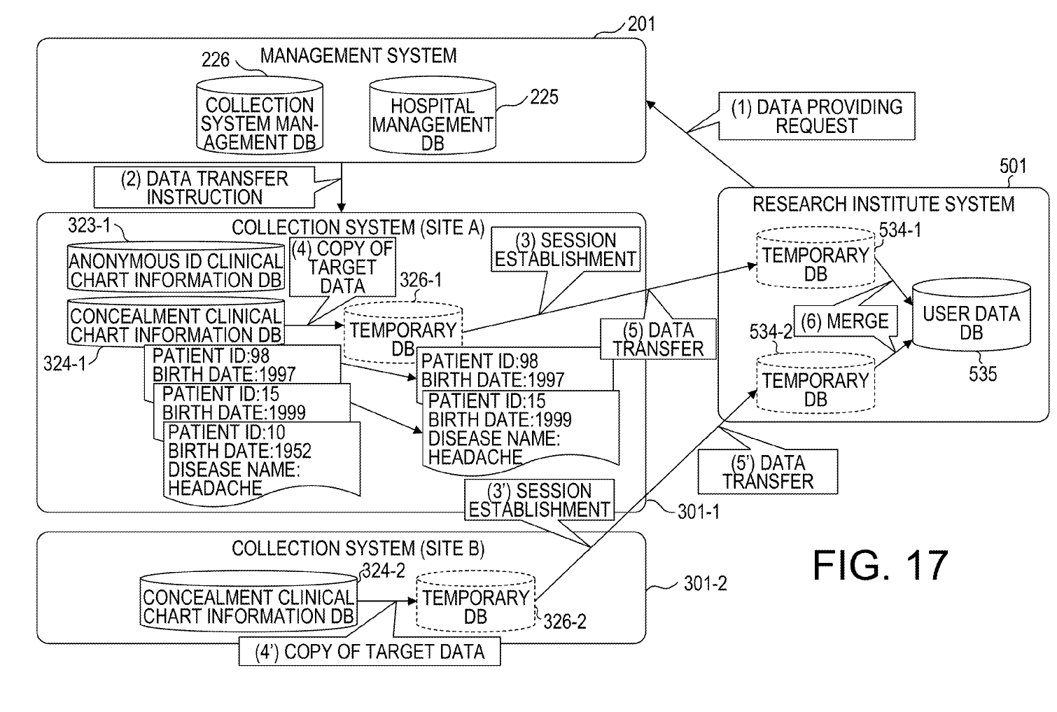

FIG. 17 is a view illustrating a process of transmitting concealed data to the research institution system according to an embodiment.

In the information processing system 100, for example, a concealed electronic clinical chart included in the concealment clinical chart information DB 324-i is transmitted to the research institution system 501 in the following sequence. (1) Because the consent of an individual is unnecessary, the research institution system 501 specifies the conditions of data to be acquired, and requests the management system 201 to provide data corresponding to the conditions. The conditions are, for example, the gender of male, the birth date from 1990 to 1999, etc. (2) The management system 201 receives a providing request from the research institution system 501 and instructs the collection system 301-i to transfer data corresponding to the conditions included in the providing request. The instruction includes the conditions received from the research institution system 501. (3) The collection system 301-1 instructed to transfer the data establishes a session for data transfer with the data management server 521 of the research institution system 501 of the transfer destination. (3′) The collection system 301-2 instructed to transfer the data establishes a session for data transfer with the data management server 521 of the research institution system 501 of the transfer destination. (4) The collection system 301-1 extracts an electronic clinical chart corresponding to the conditions received from the management system 201 from the concealment clinical chart information DB 324-1 and copies it into the temporary DB 326-1. A patient ID is also included in the temporary DB 326-1 since it is needed for the research institution system 501 to determine whether it is data of the same person when merging the data of the same person. In FIG. 17, the conditions are the birth date from 1990 to 1999, and an electronic clinical chart with the birth date from 1990 to 1999 is extracted from the concealment clinical chart information DB 324-1 and is copied into the temporary DB 326-1. (4′) The collection system 301-2 extracts an electronic clinical chart corresponding to the conditions received from the management system 201 from the concealment clinical chart information DB 324-2 and copies the electronic clinical chart to the temporary DB 326-2. (5) After copying the electronic clinical chart as the data transfer target into the temporary DB 326-1, the collection system 301-1 transmits the temporary DB 325-1 to the research institution system 501. The research institution system 501 stores the received temporary DB 326-1 as the temporary DB 534-1. (5′) After copying the electronic clinical chart as the data transfer target into the temporary DB 326-2, the collection system 301-2 transmits the temporary DB 325-2 to the research institution system 501. The research institution system 501 stores the received temporary DB 326-2 as the temporary DB 534-2. (6) The utilization system 401 merges the temporary DBs 534-1 and 534-2 into the user data DB 535 and deletes the temporary DBs 534-1 and 534-2.

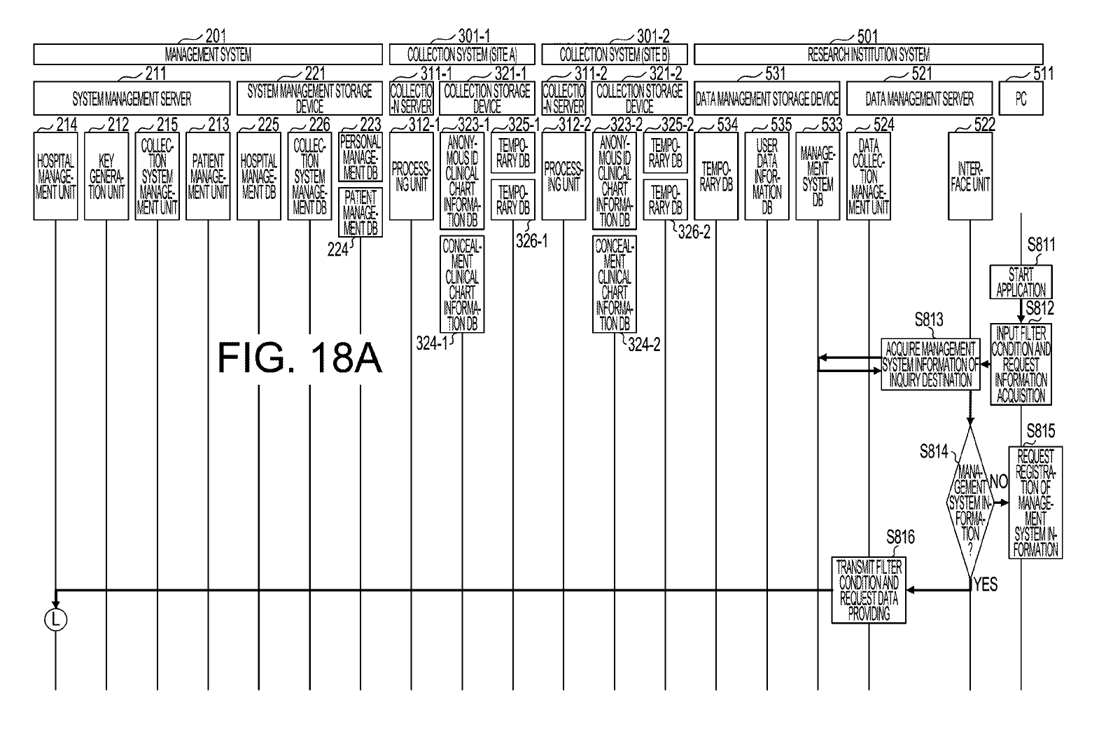

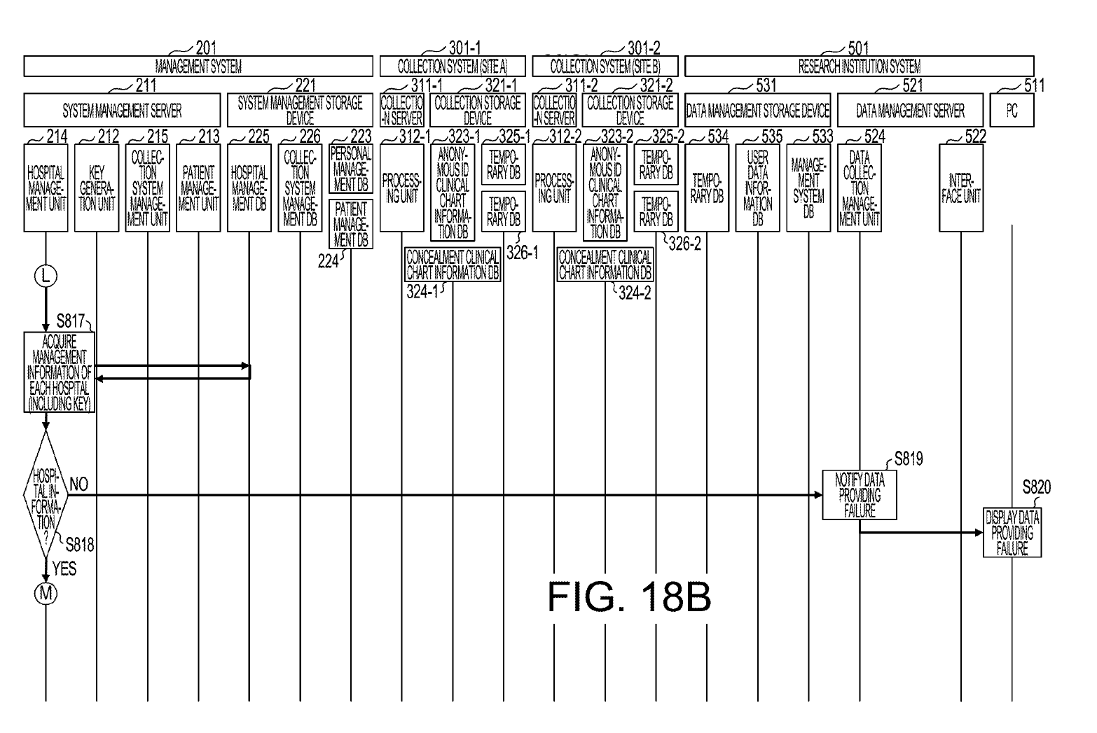

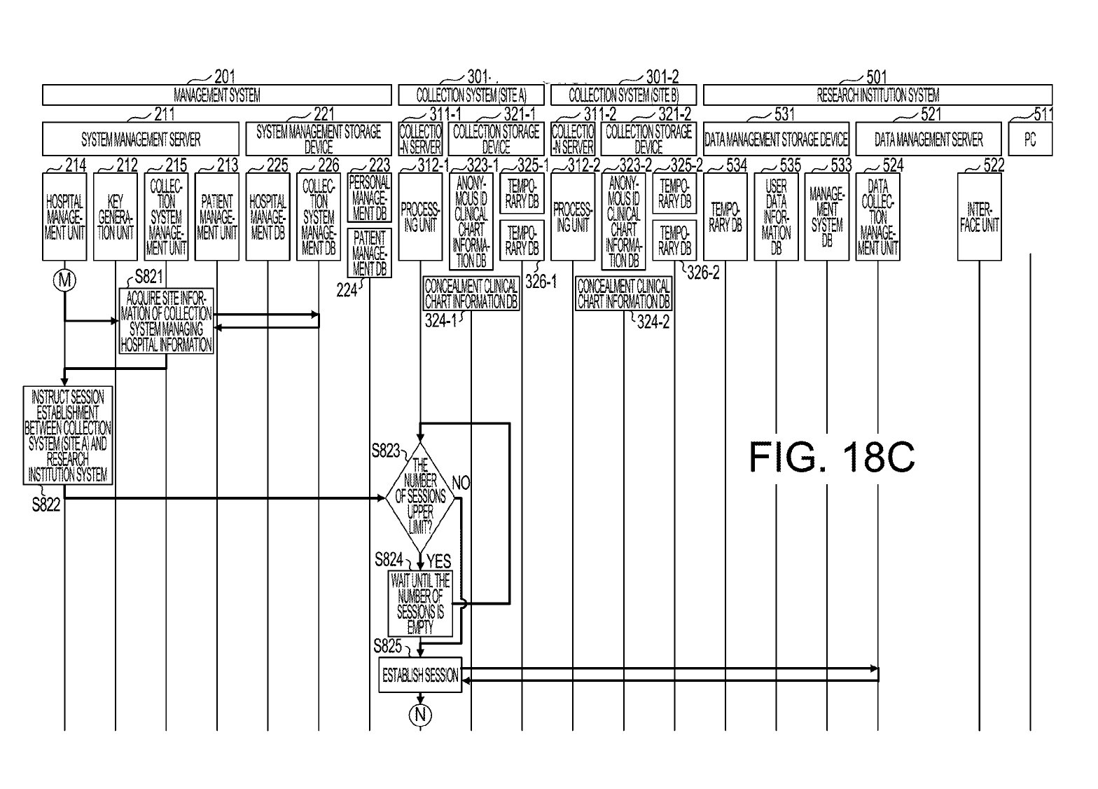

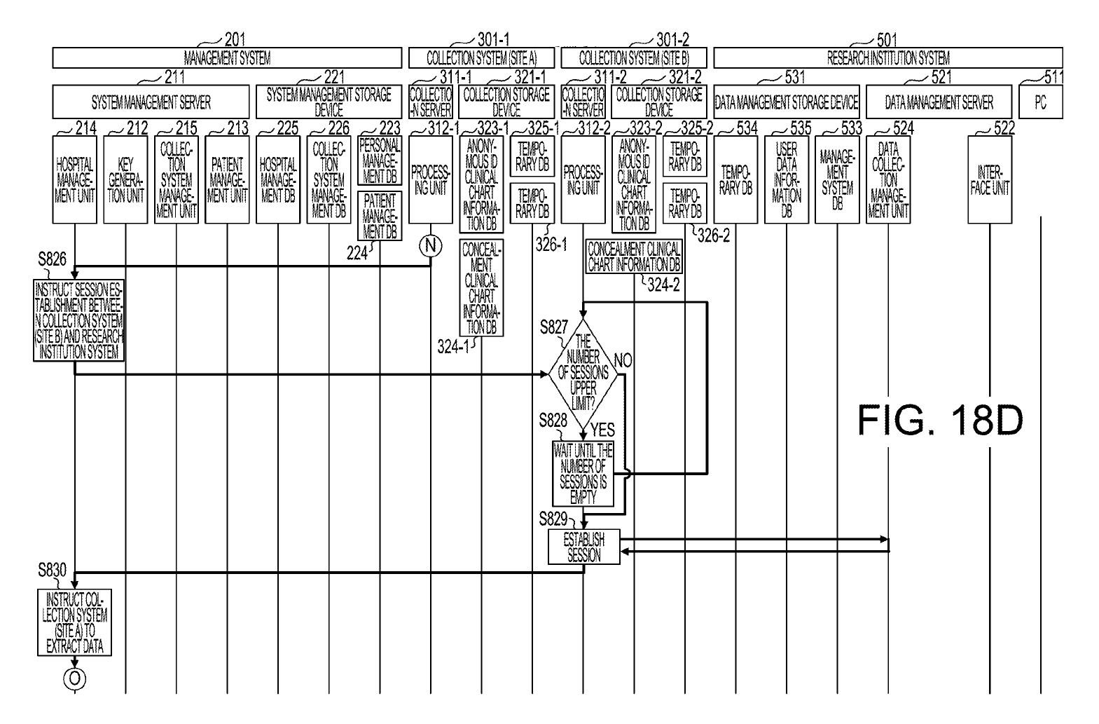

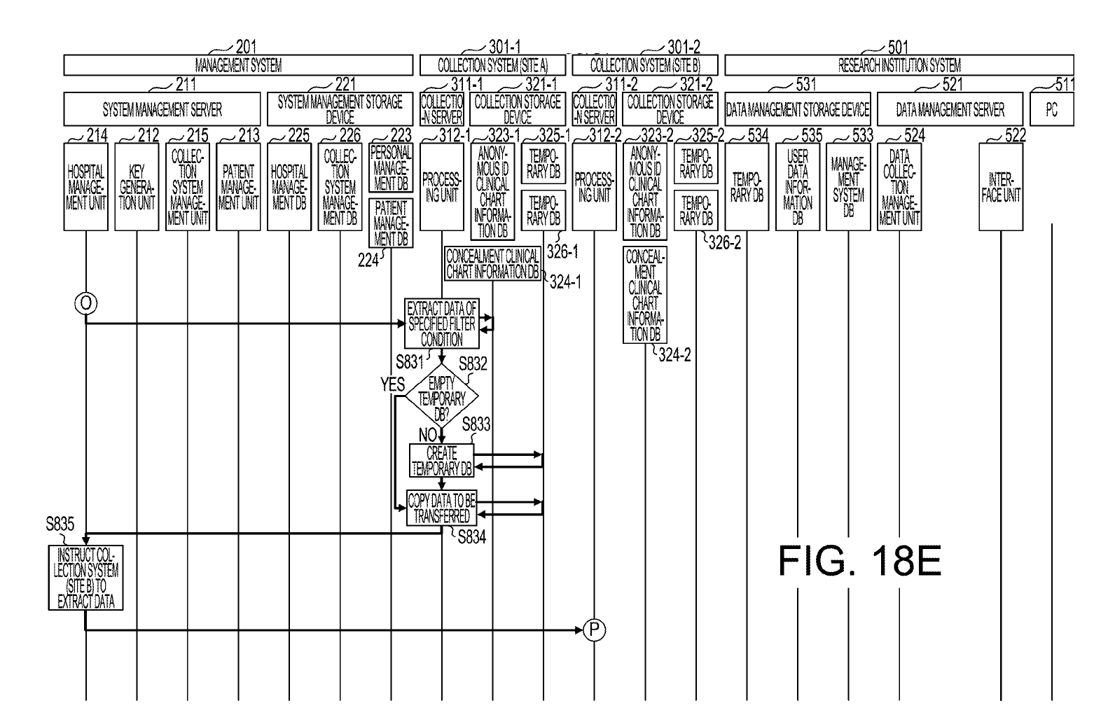

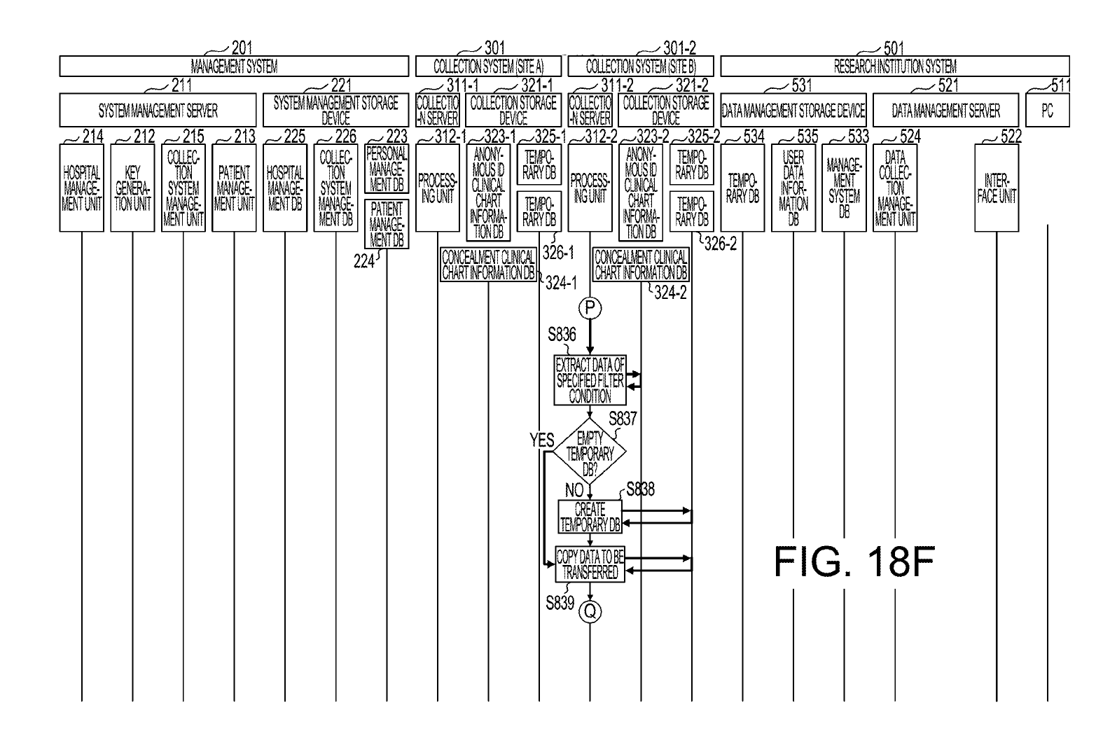

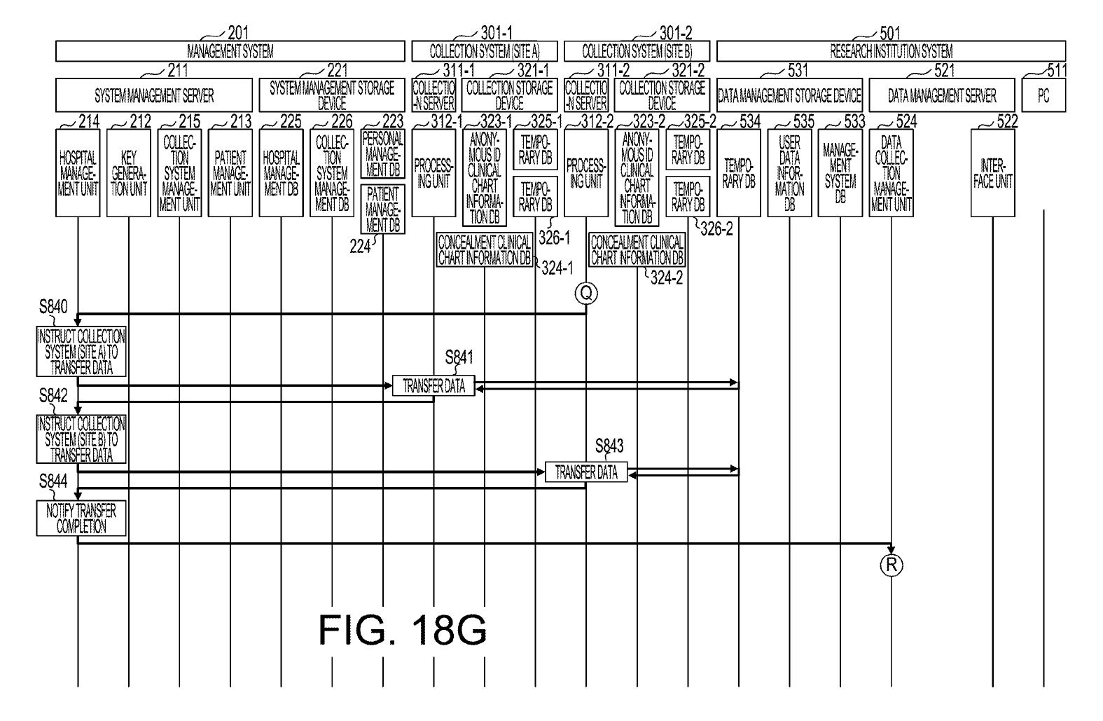

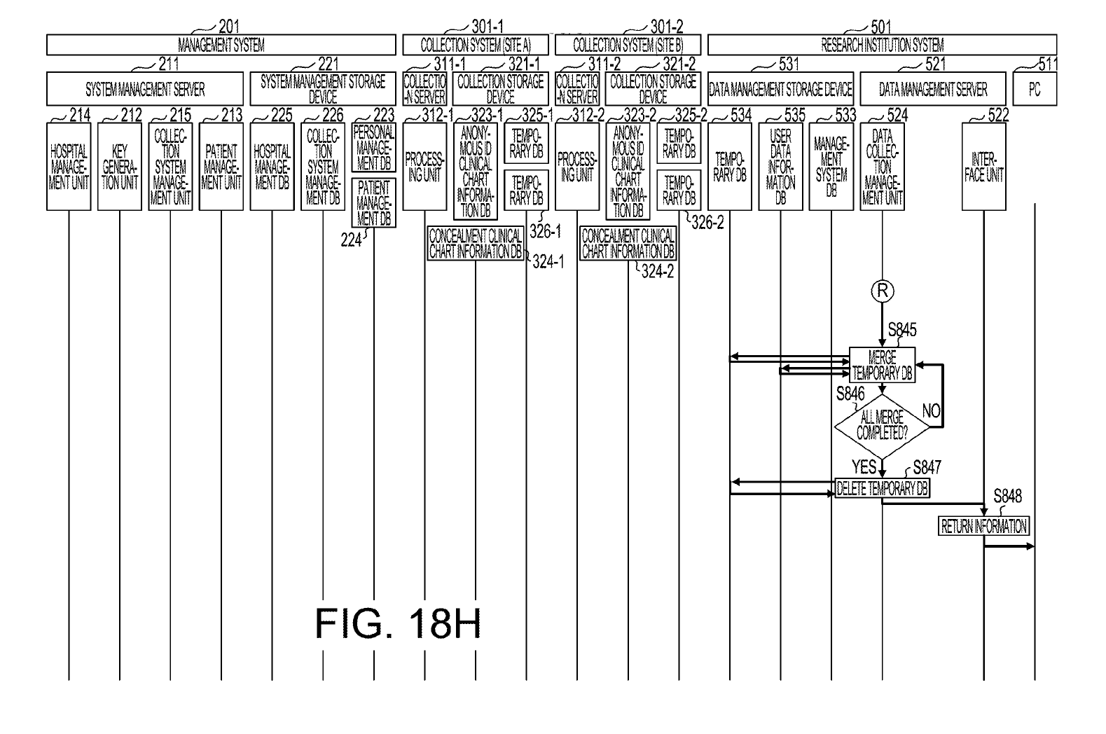

FIGS. 18A to 18H are sequence diagrams of a process of transmitting concealed data to the research institution system according to an embodiment.

In step S811, the PC 511 starts an application program installed in the PC 511.

In step S812, the PC 511 uses the application program to input a filter condition based on an operation by a researcher, and transmits a request for information acquisition to the data management server 521. The request includes the filter condition.

In step S813, the interface unit 522 refers to the management system DB 533 to acquire information (management system information) of the management system 201. The information of the management system 201 is, for example, a domain name or an IP address of the system management server 211.

In step S814, when it is determined that the management system information is not present in the management system DB 533 (when the information of the management system 201 is not acquired from the management system DB 533), the control proceeds to step S815. When it is determined that the management system information is present in the management system DB 533, the control proceeds to step S816.

In step S815, the PC 511 requests registration of the management system information, and the process is ended.

In step S816, the data collection management unit 524 transmits a data providing request including the received filter condition to the system management server 211. That is, the data collection management unit 524 requests a concealed electronic clinical chart corresponding to the filter condition. The data providing request does not include a personal ID.

In step S817, the hospital management unit 214 refers to the hospital management DB 225 to acquire information (including a hash key) of each hospital (hospital system 101-i) from the hospital management DB 225.

In step S818, when it is determined that the information of the hospital is not present in the hospital management DB 225 (when the information of each hospital is not acquired from the hospital management DB 225), the data collection management unit 524 transmits a notification of information acquisition failure to the data management server 521, and the control proceeds to step S819. When it is determined that the hospital information is present in the hospital management DB 225, the control proceeds to step S821.

In step S819, the data collection management unit 524 transmits a data providing failure notification to the PC 511.

In step S820, the PC 411 displays the data providing failure notification on the display unit (not illustrated).

In step S821, the collection system management unit 215 refers to the collection system management DB 226 to acquire information of the collection system 301-i managing data of each hospital (hospital system 101-i).

In step S822, the hospital management unit 214 instructs the collection server 311-1 of the collection system 301-1 to establish a session for data transfer between the collection server 311-1 and the data management server 521 of the research institution system 501.

In step S823, when the current number of sessions of the collection server 311-1 exceeds an upper limit, the control proceeds to step S824. When the current number of sessions of the collection server 311-1 does not exceed the upper limit, the control proceeds to S825.

In step S824, the transfer processing unit 315-1 waits until the current number of sessions of the collection server 311-1 falls below the upper limit. When the current number of sessions falls below the upper limit, the control proceeds to step S823.

In step S825, the transfer processing unit 315-1 establishes a session with the data management server 521 of the research institution system 501.

In step S826, the hospital management unit 214 instructs the collection server 311-2 of the collection system 301-2 to establish a session for data transfer between the collection server 311-2 and the data management server 521 of the research institution system 501.

In step S827, when it is determined that the current number of sessions of the collection server 311-2 exceeds the upper limit, the control proceeds to step S828. When it is determined that the current number of sessions of the collection server 311-2 does not exceed the upper limit, the control proceeds to S829.

In step S828, the transfer processing unit 315-2 waits until the current number of sessions of the collection server 311-2 falls below the upper limit. When the current number of sessions falls below the upper limit, the control proceeds to step S827.

In step S829, the transfer processing unit 315-2 establishes a session with the data management server 521 of the research institution system 501.

In step S830, the hospital management unit 214 transmits an instruction (request) for data extraction to the collection server 311-1 of the collection system 301-1. The instruction includes the filter condition received from the data management server 521. The instruction does not include a personal ID and an anonymous personal ID.

In step S831, upon receiving the instruction, the transfer processing unit 315-1 extracts an electronic clinical chart corresponding to the filter condition included in the instruction from the concealment clinical chart information DB 324-1.

In step S832, when it is determined that the empty temporary DB 326-1 is present in the storage unit 322-1, the control proceeds to step S834. When it is determined that the empty temporary DB 326-1 is not present in the storage unit 322-1, the control proceeds to step S833.

In step S833, the transfer processing unit 315-1 creates the empty temporary DB 326-1.

In step S834, the transfer processing unit 315-1 copies the extracted electronic clinical chart into the temporary DB 326-1.

In step S835, the hospital management unit 214 transmits an instruction (request) for data extraction to the collection server 311-2 of the collection system 301-2. The instruction includes the filter condition received from the data management server 521. The instruction does not include a personal ID and an anonymous personal ID.

In step S836, upon receiving the instruction, the transfer processing unit 315-2 extracts an electronic clinical chart corresponding to the filter condition included in the instruction from the concealment clinical chart information DB 324-2.

In step S837, when it is determined that the empty temporary DB 326-2 is present in the storage unit 322-2, the control proceeds to step S839. When the empty temporary DB 326-2 is not present in the storage unit 322-2, the control proceeds to step S838.

In step S838, the transfer processing unit 315-2 creates the empty temporary DB 326-2.

In step S839, the transfer processing unit 315-2 copies the extracted electronic clinical chart into the temporary DB 326-2.

In step S840, the hospital management unit 214 transmits an instruction of data transfer to the collection server 311-1 of the collection system 301-1.

In step S841, the transfer processing unit 315-1 transmits the temporary DB 326-1 to the data management server 521, and the data collection management unit 524 stores the received temporary DB 326-1 as the temporary DB 534-1 in the storage unit 532.

In step S842, the hospital management unit 214 transmits an instruction of data transfer to the collection server 311-2 of the collection system 301-2.

In step S843, the transfer processing unit 315-2 transmits the temporary DB 326-2 to the data management server 521, and the data collection management unit 524 stores the received temporary DB 326-2 as the temporary DB 534-2 in the storage unit 532.

In step S844, the hospital management unit 214 notifies the data management server 521 of the completion of the transfer.

In step S845, the data collection management unit 524 merges an unmerged temporary DB 534-i into the user data DB 535.

In step S846, when it is determined that all temporary DBs 534-i have been merged into the user data DB 535, the control proceeds to step S847. When it is determined that there is an unmerged temporary DB 534-i, the control returns to step S845.

In step S847, the data collection management unit 524 deletes the temporary DB 534-i.

In step S848, the interface unit 522 acquires an electronic clinical chart corresponding to the filter condition from the user data DB 535 and transmits it to the PC 411.

With the information processing system 100 according to an embodiment, it is possible to provide data corresponding to each of the service utilization system 401 and the research institution system 501. That is, with the information processing system 100 according to an embodiment, it is possible to transmit non-concealed data to the service utilization system 401 that uses the non-concealed data and transmit concealed data to the research institution system 501 that uses the concealed data.

With the information processing system 100 according to an embodiment, since even the same patient in the anonymous ID clinical chart information DB 323-i is managed with different anonymous patient IDs for different hospitals, except for a regular route through the management system, data of a specific person may not be merged, thereby reducing a security risk of specifying a person.

In the concealment clinical chart information DB 324-i, since information specifying a person is concealed, merging the data of the same person is unproblematic. With the information processing system 100 according to an embodiment, since the data of the same person is managed with the same patient ID in the concealment clinical chart information DB 324-I, it is possible to determine whether or not a plurality of pieces of data is the data of the same person and to merge the data of the same person.

With the information processing system 100 according to an embodiment, since communication including a personal ID that can uniquely specify a user is performed only between the hospital system 101-i and the management system 201 and between the service utilization system 401 and the management system 201, it is possible to reduce the security risk.

In the information processing system 100 of FIG. 1, the information providing institution may be an institution other than a hospital providing patient medical examination information. For example, the information providing institution may be a shop that provides purchase information of a customer, a school that provides student's score information, an educational institution such as a preparatory school, or a financial institution such as a bank that provides customer's deposit balance, transaction result, etc.

In the case where the information providing institution is a shop, customer purchase information is collected as personal information and is provided to another shop, which is an information utilization institution, under the consent of the customer. In addition, concealed customer purchase information is provided to a research company which is an information analysis institution for analyzing customer preferences and the like.

In the case where the information providing institution is an educational institution, student score information is collected as personal information and is provided to another educational institution, which is an information utilization institution, under the consent of the student. In addition, concealed student score information is provided to a research company which is an information analysis institution for analyzing trends and the like for each subject.

In a case where the information providing institution is a financial institution, information such as customer's deposit balance, transaction results, etc. is collected as personal information and is provided to another financial institution, which is an information utilization institution, under the consent of the customer. In addition, information on concealed customer's deposit balance, transaction results, etc. is provided to a research company which is an information analysis institution for analyzing the usage situation of loans.

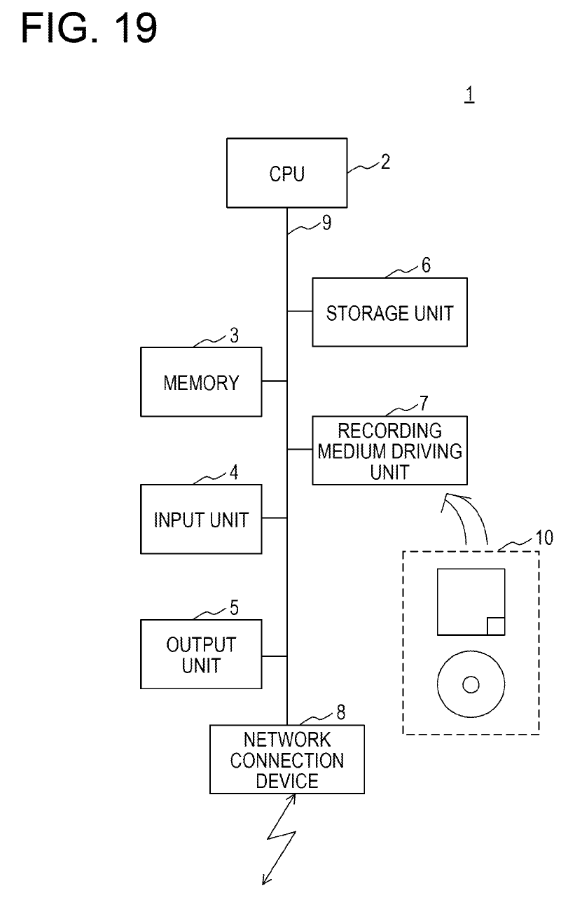

FIG. 19 is a view illustrating the configuration of an information processing apparatus (computer). The doctor PC 111-i, the electronic clinical chart management server 121-i, the system management server 211, the collection server 311-i, the PCs 411 and 511, and the data management servers 421 and 521 of the embodiment may be implemented by the information processing apparatus (computer) 1 as illustrated in FIG. 19, for example.

The information processing apparatus 1 includes a central processing unit (CPU) 2, a memory 3, an input device 4, an output device 5, a storage unit 6, a recording medium driving unit 7, and a network connection device 8, which are interconnected by a bus 9.

The CPU 2 is a central processing unit that controls the overall operation of the information processing apparatus 1. The CPU 2 operates as the processing unit 121-i, the key generation unit 212, the patient management unit 213, the hospital management unit 214, the collection system management unit 215, the processing unit 312-i, the web service unit 422, the user management unit 423, the data collection management unit 424, the interface unit 522, and the data collection management unit 524.

The memory 3 is a memory such as a read only memory (ROM) or a random access memory (RAM) that temporarily stores a program or data stored in the storage unit 6 (or the portable recording medium 10) at the time of execution of the program. The CPU 2 executes the program using the memory 3, thereby executing the various processes described above.

In this case, a program code itself read from the portable recording medium 10 or the like implements the functions of the embodiment.

The input device 4 is used to input instructions and information from a user or an operator and acquire data used in the information processing apparatus 1. The input device 4 is, for example, a keyboard, a mouse, a touch panel, a camera, a sensor, or the like.

The output device 5 is a device that outputs an inquiry or a processing result to the user or the operator or operates under the control of the CPU 2. The output device 5 is, for example, a display, a printer or the like.

The storage unit 6 is, for example, a magnetic disk device, an optical disk device, a tape device, or the like. The information processing apparatus 1 stores the above-described programs and data in the storage unit 6 and reads and uses them into the memory 3 as necessary. The memory 3 and the storage unit 6 correspond to the storage unit 125-i.

The recording medium driving unit 7 drives the portable recording medium 10 and accesses the recorded contents. The portable recording medium may be any computer-readable recording medium such as a memory card, a flexible disk, a compact disk read only memory (CD-ROM), an optical disk, a magneto-optical disk, or the like. A user stores the above-described program and data in the portable recording medium 10 and reads and uses them into the memory 3 as necessary.

The network connection device 8 is a communication interface which is connected to an arbitrary communication network such as a local area network (LAN) or a wide area network (WAN) and performs data conversion accompanying communication. The network connection device 8 transmits data to a device connected via a communication network or receives data from a device connected via a communication network.

It is not necessary for the information processing apparatus 1 to include all the constituent elements in FIG. 19 and it is also possible to omit some constituent elements depending on usages or conditions.

All examples and conditional language recited herein are intended for pedagogical purposes to aid the reader in understanding the invention and the concepts contributed by the inventor to furthering the art, and are to be construed as being without limitation to such specifically recited examples and conditions, nor does the organization of such examples in the specification relate to an illustrating of the superiority and inferiority of the invention. Although the embodiments of the present invention have been described in detail, it should be understood that the various changes, substitutions, and alterations could be made hereto without departing from the spirit and scope of the invention.

What is claimed is:

1. An information processing apparatus comprising:
a memory; and
a processor coupled to the memory and configured to:
receive, from a management device, a first request provided from a first device that requests personal information associated with a first identifier, the first request including a second identifier obtained by converting the first identifier, and a second request which includes an extraction condition and is provided from a second device that requests concealed personal information corresponding to the extraction condition;
extract personal information associated with the second identifier from a storage device that stores a plurality of personal information associated respectively with a plurality of second identifiers, when the first request is received, and extract concealed personal information corresponding to the extraction condition from the storage device that stores a plurality of concealed personal information, when the second request is received; and
transmit the extracted personal information associated with the second identifier to the first device, when the first request is received, and transmit the extracted concealed personal information corresponding to the extraction condition to the second device, when the second request is received.

2. The information processing apparatus according to claim 1, wherein the processor is further configured to:
transmit the first identifier from the first device to the management device; and
hash the second identifier with a hash key associated with a personal information management device that stores personal information associated with the first identifier in the management device, or, if there is a plurality of personal information management devices, hash the second identifier with different hash keys having different values associated with different personal information.

3. An information processing system comprising:
a first device configured to request personal information associated with a first identifier;
a second device configured to request concealed personal information corresponding to an extraction condition;
a management device configured to generate a second identifier into which the first identifier is converted; and
an information processing apparatus configured to transmit personal information associated with the second identifier and the concealed personal information corresponding to the extraction condition,
wherein the first device transmits a first request including the first identifier to the management device and the second device transmits a second request including the extraction condition to the management device,
the management device generates the second identifier when the first request is received, and transmits a third request including the second identifier, and transmits a fourth request including the extraction condition to the information processing apparatus when the second request is received, and
the information processing apparatus includes:
a memory; and
a processor coupled to the memory and configured to:
receive the third request and the fourth request;
extract personal information associated with the second identifier from a storage device that stores a plurality of personal information associated respectively with a plurality of second identifiers, when the third request is received, and extract concealed personal information corresponding to the extraction condition from the storage device that stores a plurality of concealed personal information, when the fourth request is received; and
transmit the extracted personal information associated with the second identifier to the first device, when the third request is received, and transmit the extracted concealed personal information to the second device, when the fourth request is received.

4. The information processing system according to claim 3, wherein the management device stores first information indicating the association between the first identifier and a personal information management device storing personal information associated with the first identifier, and stores second information indicating the association between the personal information management device and a hash key associated with the personal information management device, or, when there is a plurality of personal information management devices, stores second information indicating the association between the personal information management device and different hash keys having different values associated with different personal information, and
when the first request is received, the management device specifies a personal information management device storing the first identifier, and the hash key associated with the personal information management device, based on the first information and the second information, generates the second identifier based on the first identifier and the specified hash key, and transmits a third request including the second identifier to the information processing apparatus.

5. A non-transitory computer-readable medium having stored therein a program that causes a computer to perform a process, the process comprising:
receiving, from a management device, a first request including a second identifier into which a first identifier is converted and a second request that includes an extraction condition and but does not include the second identifier;
when the first request is received, extracting personal information associated with the second identifier from a storage device storing a plurality of personal information associated respectively with a plurality of identifiers, and transmitting the personal information associated with the second identifier to a first device requesting personal information associated with the first identifier; and
when the second request is received, extracting concealed personal information corresponding to the extraction condition from a plurality of concealed personal information stored in the storage device, and transmitting concealed personal information corresponding to the extraction condition to a second device that requests the concealed personal information corresponding to the extraction condition.

6. The non-transitory computer-readable medium according to claim 5, wherein the process further comprising:
transmitting the first identifier from the first device to the management device; and
hashing the second identifier with a hash key associated with a personal information management device that stores personal information associated with the first identifier in the management device, or, when there is a plurality of personal information management devices, hashing the second identifier with different hash keys having different values associated with different personal information.
